(19) United States
(12) Patent Application Publication
(10) Pub. No.: US 2016/0090574 A1
(43) Pub. Date: Mar. 31, 2016

(54) A PROCESS FOR THE PRODUCTION OF ADENOVIRUS

(71) Applicant: PSIOXUS THERAPUETICS LIMITED, Abingdon Oxfordshire (GB)

(72) Inventors: Kerry Fisher, Oxfordshire (GB); Jeetendra Bhatia, Abingdon, Oxfordshire (GB); Brian Robert Champion, Abingdon, Oxfordshire (GB)

(21) Appl. No.: 14/769,389

(22) PCT Filed: Feb. 28, 2014
(86) PCT No.: PCT/EP2014/053987

§ 371 Date: Aug. 20, 2015

Related U.S. Application Data
(60) Provisional application No. 61/770,513, filed on Feb. 28, 2013.

(51) Int. Cl. C12N 7/00

(52) U.S. Cl. CPC C12N 7/00; C12N 2710/10032; C12N 2710/10021; C12N 2710/10052; C12N 2710/10051

(57) ABSTRACT

The present disclosure relates to a process for the manufacture of adenoviruses wherein the process comprises culturing mammalian cells infected with the adenovirus in the presence of media suitable for supporting the cells such that the virus replicates, wherein the cells are capable of supporting viral replication, and at the end of the culturing period isolating from the media the adenovirus by filtering wherein the isolation of virus is not subsequent to a cell lysis step and to viruses obtainable from the process.

The present disclosure relates to a method for the manufacture of certain adenoviruses, for example chimeric adenoviruses, in particular replication competent adenoviruses, and the viral product obtained therefrom.

### BACKGROUND

At the present time the pharmaceutical field is on the edge of realising the potential of viruses as therapeutics for human use. To date a virus derived from ONXY-15 (ONYX Pharmaceuticals and acquired by Shanghai Sunway Biotech) is approved for use in head and neck cancer in a limited number of countries. However, there are a number of viruses currently in the clinic, which should hopefully result in some of these being registered for use in humans.

A number of virus therapies are based on adenoviruses, for example ColoAd1 is a chimeric oncolytic adenovirus (WO 2005/118825) currently in clinical trials for the treatment of colorectal cancer.

These adenoviral based therapeutic agents need to be manufactured in quantities suitable for supporting both the clinical trials and demand after registration and under conditions that adhere to good manufacturing practice (GMP).

As part of the manufacturing process, the viruses are propagated in mammalian cells in vitro, for example in a cell suspension culture. The virus is recovered from these cells by cell lysis and subsequent purification. FIG. 1 is an extract from Kamen & Henry 2004 (J Gene Med. 6: pages 184-192) showing a schematic diagram of the processes involved manufacture of the GMP grade adenovirus. Notably, after viral replication, the cells are lysed.

Contaminating DNA from the cells after lysis is a significant problem and must be removed as far as possible from the therapeutic adenoviral product. This is described in detail in the application WO 2011/045381, which describes lysing the cells, fragmenting or precipitating the DNA within the cell suspension and clarifying the same, employing tangential flow. DNA digestion with DNAse is also shown as the third step in FIG. 1.

Much of the work in the field of GMP manufacture of adenoviruses has been performed on Ad5. The prior art indicates that for a batch process a maximum virus titre is achieved around 40 hours post infection and thereafter cell death starts to occur. Furthermore, concerns about reduction in viral infectivity, which is a measure of the activity of the virus produced, are usually addressed by keeping processing times to a minimum in any GMP process.

In short, developing a successful recombinant adenovirus process requires a detailed understanding of basic host cell line physiology and metabolism; the recombinant virus, and the interaction between the cell line and the virus. Essentially the process requires adaptation depending on the particular type of virus or viral vector.

Surprisingly the present inventors have established that chimeric oncolytic adenovirus can be prepared by a process that isolates the virus from the cell media and that avoids the necessity to lyse the cells and thus significantly reduces the starting level of DNA contamination in the viral product.

### SUMMARY OF THE INVENTION

Thus the present disclosure provides a process for the manufacture of a chimeric oncolytic adenovirus having a genome comprising an E2B region, wherein said E2B region comprises a nucleic acid sequence from a first adenoviral serotype and a nucleic acid sequence from a second distinct adenoviral serotype; wherein said first and second serotypes are each independently selected from the adenoviral subgroups B, C, D, E, F or G wherein the process comprises the steps:
a. culturing mammalian cells infected with the adenovirus in the presence of media suitable for supporting the cells such that the virus replicates, wherein the cells are capable of supporting viral replication, and
b. at the end of the culturing period isolating from the media the adenovirus from step a) by filtering
   wherein the isolation of virus is not subsequent to a cell lysis step.

Also provided is a process for the manufacture of adenovirus having a fibre and hexon of subgroup B (such as Ad11, in particular Ad11p also known as the Slobitski strain) wherein part of the E4 region is deleted said process comprises the steps:
a. culturing mammalian cells infected with the adenovirus in the presence of media suitable for supporting the cells such that the virus replicates, wherein the cells are capable of supporting viral replication, and
b. at the end of the culturing period isolating from the media the virus from step a) by filtering
   wherein the isolation of virus is not subsequent to a cell lysis step. In one embodiment the virus is replication competent or replication deficient.

In one embodiment the adenovirus has part or all of the E3 region deleted.

Surprisingly the present inventors have also found that the process can successfully be extended to wild-type Ad11 viruses, such as Ad11p, also to viruses having a fibre and hexon from Ad11, including Ad11p.

### BRIEF DESCRIPTION OF THE FIGURES

FIG. 1 is an extract from Kamen and Henry 2004 (J Gene Med. 6: S184-192) showing a schematic diagram of the processes involved manufacture of the GMP grade adenovirus.

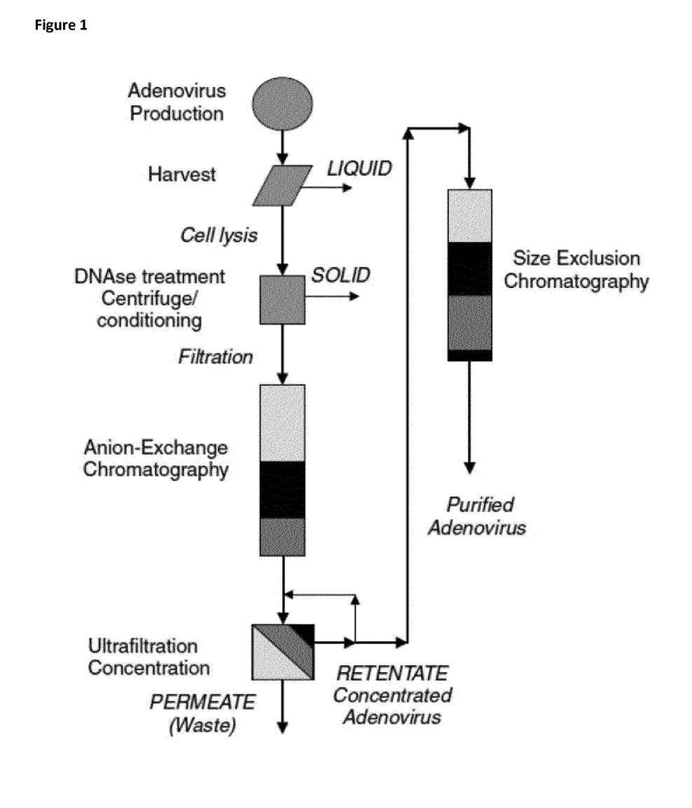

FIG. 2 shows the proportion of infectious ColoAd1 particles associated with the cells and supernatant (SN) of suspension HEK293s infected at MOI 10.

FIG. 3 shows the proportion of infectious ColoAd1 particles associated with the cells and supernatant (SN) of adherent HEK293s infected at MOI 10 (multiplicity of infection 10).

FIG. 4 shows total viral particle amounts of suspension HEK293 culture in infection condition testing

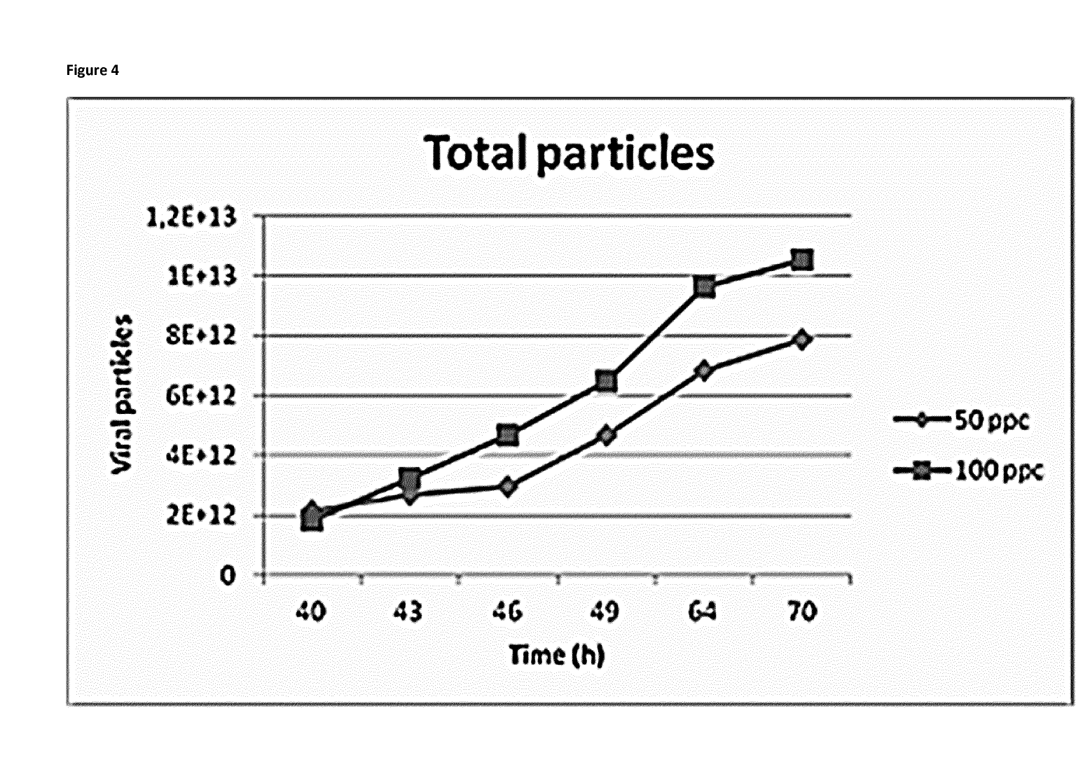

FIG. 5 Visualisation of cellular and viral DNA in the cell lysate (Lysate) or supernatant (SN) of ColoAd1 infected HEK293 cells at 40 hrs, 46 hrs and 70 hrs post-infection

FIG. 6 A—Virus distribution (CVL or supernatant),

B—Total virus production (vp/cell) and

C—Cell viability at each time point post-infection for ColoAd1.

FIG. 7 A—Virus distribution (CVL or supernatant),

B—Total virus production (vp/cell), and

C—Cell viability at each time point post-infection for NG135.

FIG. 8 A—Virus distribution (CVL or supernatant),

B—Total virus production (vp/cell), and

C—Cell viability at each time point post-infection for NG76.

FIG. 9 A—Virus distribution (CVL or supernatant),

B—Total virus production (vp/cell) and

C—Cell viability at each time point post-infection for wild-type Ad5.

FIG. 10 A—Virus distribution (CVL or supernatant),

B—Total virus production (vp/cell) and

C—Cell viability at each time point post-infection for wild-type Ad11p.

NG135 as employed herein refers to a derivative of the ColoAd1 virus with a transgene inserted. The transgene is a full length antibody. NG135 is SEQ ID 1 with an added transgene cassette. NG76 as employed herein refers to a derivative of the ColoAd1 virus with a transgene inserted. The transgene is a ScFv antibody fragment. NG76 is SEQ ID 1 with an added transgene cassette.

### DETAILED DESCRIPTION OF THE DISCLOSURE

A process for the manufacture of a chimeric oncolytic virus as employed herein is intended to refer to a process wherein the virus is replicated and thus the number of viral particles is increased. In particular the manufacturing is to provide sufficient numbers of viral particles to formulate a therapeutic product, for example in the range 1-9×10⁵ to 1-9×10²⁰ or more particles may be produced, such as in the range of 1-9×10⁸ to 1-9×10¹⁵ viral particles, in particular 1 to 9×10¹⁰ or 1-9×10¹⁵ viral particles may be produced from a 10 L batch.

Part of the E4 region is deleted as employed herein means that at least part, for example in the range 1 to 99% of the E4 region is deleted, such as 2, 3, 4, 5, 6, 7, 8, 9, 10, 15, 20, 25, 30, 35, 40, 45, 50, 55, 60, 65, 70, 75, 80, 85, 90, 91, 92, 93, 94 95, 96, 97 or 98% deleted.

“Derived from” as employed herein refers to, for example where a DNA fragment is taken from an adenovirus or corresponds to a sequence originally found in an adenovirus. This language is not intended to limit how the sequence was obtained, for example a sequence employed in a virus according to the present disclosure may be synthesised.

In one embodiment the derivative has 100% sequence identity over its full length to the original DNA sequence.

In one embodiment the derivative has 95, 96, 97, 98 or 99% identity or similarity to the original DNA sequence.

In one embodiment the derivative hybridises under stringent conditions to the original DNA sequence.

As used herein, “stringency” typically occurs in a range from about Tm (melting temperature)-50 C (5° below the Tm of the probe) to about 20° C. to 25° C. below Tm. As will be understood by those of skill in the art, a stringent hybridization can be used to identify or detect identical polynucleotide sequences or to identify or detect similar or related polynucleotide sequences. As herein used, the term “stringent conditions” means hybridization will generally occur if there is at least 95%, such as at least 97% identity between the sequences.

As used herein, “hybridization” as used herein, shall include “any process by which a polynucleotide strand joins with a complementary strand through base pairing” (Coombs, J., Dictionary of Biotechnology, Stockton Press, New York, N.Y., 1994).

“Wherein the isolation is not subsequent to a cell lysis step” as employed herein is intended to refer to the fact the manufacturing process does not comprise a specific lysis step. That is to say a step where the conditions are designed to lyse all or most of the cells in the culture. For example, the virus is isolated from the supernatant.

Most as employed herein refers to a large majority, for example 80, 90, 91, 92, 93, 94, 95, 96, 97, 98 or 99%.

“At the end of the culturing period” as employed herein refers to at the end of a period over which the virus in the infected cells has been allowed to replicate. End refers to a selected point in time selected for harvesting. End as employed herein is not definitive end-point. In one embodiment the end-point is chosen to follow a sufficient period of cultivation for the replicated virus or a significant proportion thereof to be released into the medium or supernatant. In one embodiment the harvesting occurs at multiple time points or is ongoing after it is initiated.

Advantageously, the present process may simplify downstream processing of the virus because of the lower starting concentration of contaminating DNA from the cells because a cell lysis step is avoided. This may result in cost savings because reagents, equipment and time employed in downstream processing may be reduced. It may also result in greater purity with lower end concentrations of contaminating DNA and/or a lower concentration of large fragments of contaminating DNA.

Furthermore, virus exposure to cell enzymes is minimised by avoiding cell lysis, which minimises the exposure of the virus to potential degradants, such as nucleases from the cell. This may result in higher virus stability and/or potency as measured, for example by infectivity.

The use of benzonase to degrade cellular DNA may also be avoided or reduced if desired, which may be advantageous. In particular, removal of the benzonase and testing to show the absence of residual benzonase can be avoided.

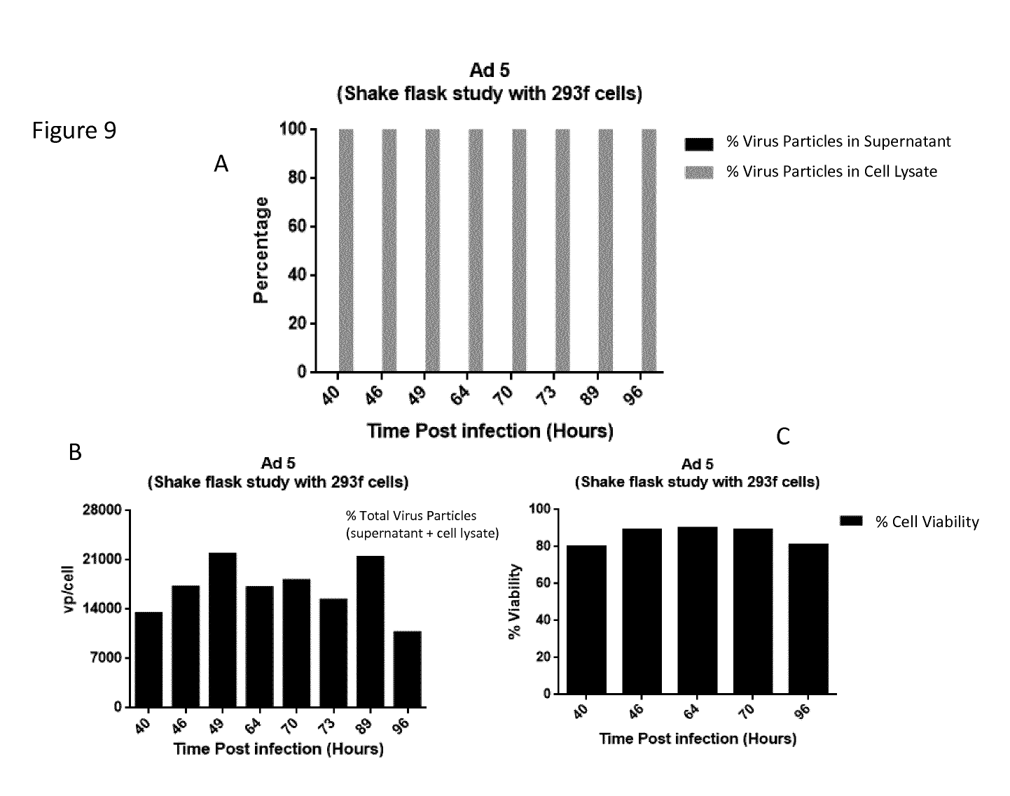

Interestingly, after exiting the cells the virus of the present disclosure does not adhere to the cells and so can be readily recovered from the supernatant. This may be a phenomenon which is characteristic of the oncolytic viruses described herein which facilitates the current process. In contrast, wild-type Ad5 is thought to adhere to cells. In fact, results have shown that substantially no wild-type Ad5, viral particles are present in the supernatant (see FIG. 9 & table 6).

Whilst not wishing to be bound by theory, in one embodiment the ability to exit the cell and not adhere thereto, may be associated with the chimeric E2B region.

In one embodiment the ability to exit the cell may be associated with a small viral genome and/or a partial deletion in the E4 and/or E3 region.

In one embodiment viruses of the present disclosure further comprise a transgene.

In one embodiment the lack of adherence to the cells may be related to the hexon and fibre of the oncolytic virus.

Oncolytic viruses are those which preferentially infect cancer cells and hasten cell death, for example by lysis of same, or selectively replicate in the cancer cells.

Viruses which preferentially infect cancer cells are viruses which show a higher rate of infecting cancer cells when compared to normal healthy cells.

A chimeric adenovirus of the present disclosure can be evaluated for its preference for a specific tumor type by examination of its lytic potential in a panel of tumor cells, for example colon tumor cell lines include HT-29, DLD-1, LS174T, LS1034, SW403, HCT116, SW48, and Colo320DM. Any available colon tumor cell lines would be equally useful for such an evaluation.

Prostate cell lines include DU145 and PC-3 cells. Pancreatic cell lines include Panc-1 cells. Breast tumor cell lines include MDA231 cell line and ovarian cell lines include the OVCAR-3 cell line. Hemopoietic cell lines include, but are not limited to, the Raji and Daudi B-lymphoid cells, K562 erythroblastoid cells, U937 myeloid cells, and HSB2 T-lymphoid cells. Other available tumor cell lines are equally useful.

Oncolytic viruses including those which are non-chimeric, for example Ad11, such as Ad11p can similarly be evaluated in these cell lines.

Viruses which selectively replicate in cancer cells are those which require a gene or protein which is upregulated in a cancer cell to replicate, such as a p53 gene.

In one embodiment the chimeric oncolytic virus is apoptotic, that is hastens programmed cell death.

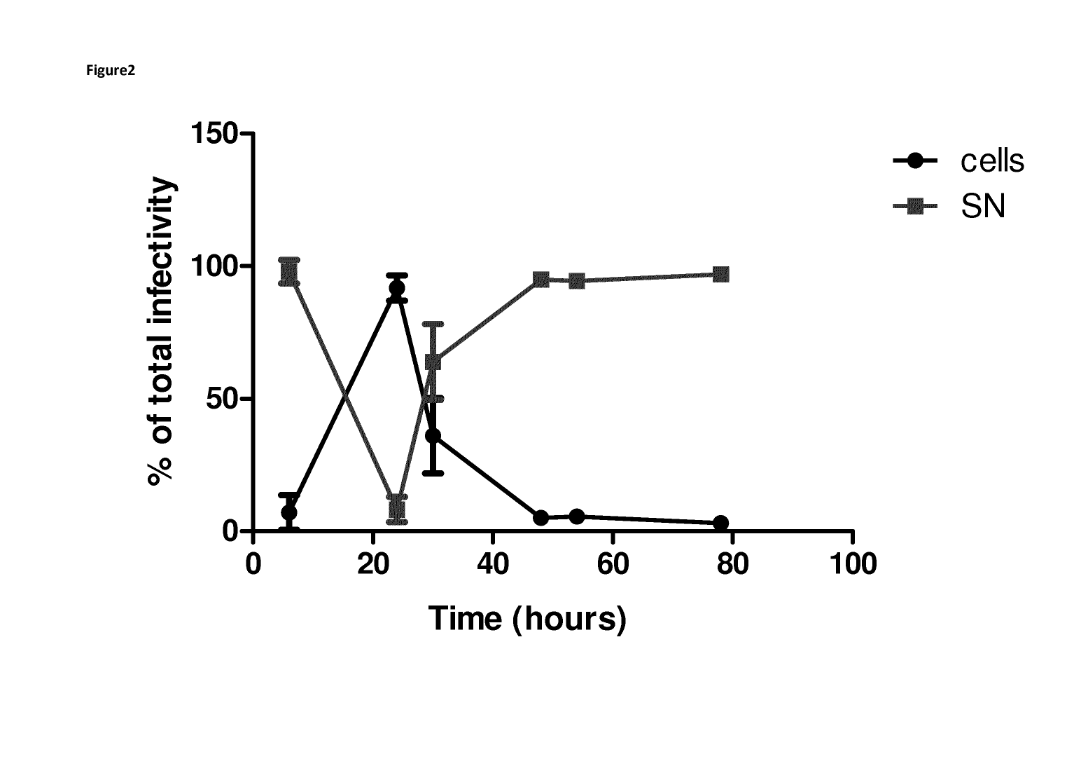

In one embodiment the chimeric oncolytic virus is cytolytic. The cytolytic activity of chimeric oncolytic adenoviruses of the disclosure can be determined in representative tumor cell lines and the data converted to a measurement of potency, for example with an adenovirus belonging to subgroup C, preferably Ad5, being used as a standard (i.e. given a potency of 1). A suitable method for determining cytolytic activity is an MTS assay (see Example 4, FIG. 2 of WO 2005/118825 incorporated herein by reference).

In one embodiment the chimeric oncolytic adenovirus of the present disclosure causes cell necrosis.

In one embodiment the chimeric oncolytic virus has an enhanced therapeutic index for cancer cells.

Therapeutic index” or “therapeutic window” refers to a number indicating the oncolytic potential of a given adenovirus which may be determined by dividing the potency of the chimeric oncolytic adenovirus in a relevant cancer cell line by the potency of the same adenovirus in a normal (i.e. non-cancerous) cell line.

In one embodiment the chimeric oncolytic virus has an enhanced therapeutic index in one or more cancer cells selected from the group comprising colon cancer cells, breast cancer cells, head and neck cancers, pancreatic cancer cells, ovarian cancer cells, hemopoietic tumor cells, leukemic cells, glioma cells, prostate cancer cells, lung cancer cells, melanoma cells, sarcoma cells, liver cancer cells, renal cancer cells, bladder cancer cells and metastatic cancer cells.

A chimeric oncolytic adenovirus as employed herein refers to an adenovirus comprising an E2B region which has DNA sequence derived from at least two distinct adenovirus serotypes and wherein the virus is oncolytic.

There are currently about 56 adenovirus serotypes. Table 1 shows the division of adenovirus serotypes:

| Subgroup | Adenoviral Serotype |
|---|---|
| A | 12, 18, 31 |
| B | 3, 7, 11, 14, 16, 21, 34, 35, 50, 55 |
| C | 1, 2, 5, 6 |
| D | 8-10, 13, 15, 17, 19, 20, 22-30, 32, 33, 36-39, |
| | 42-51, 53, 54, 56 |
| E | 4 |
| F | 40, 41 |
| G | 52 |

The E2B region is a known region in adenoviruses and represents about 18% of the viral genome. It is thought to encode protein IVa2, DNA polymerase and terminal protein. In the Slobitski strain of Ad11 (referred to as Ad11p) these proteins are encoded at positions 5588-3964, 8435-5067 and 10342-8438 respectively in the genomic sequence and the E2B region runs from 10342-3950. The exact position of the E2B region may change in other serotypes but the function is conserved in all human adenovirus genomes examined to date as they all have the same general organisation.

In one embodiment the virus of the present disclosure, such as a chimeric oncolytic virus has a subgroup B hexon.

In one embodiment the virus of the disclosure, such as a chimeric oncolytic virus has an Ad11 hexon, such as an A11p hexon.

In one embodiment the virus of the disclosure, such as a chimeric oncolytic virus has a subgroup B fibre.

In one the virus of the disclosure, such as a chimeric oncolytic virus has an Ad11 fibre, such as an A11p fibre.

In one embodiment the virus of the disclosure, such as a chimeric oncolytic virus has fibre and hexon proteins from the same serotype, for example a subgroup B adenovirus, such as Ad11, in particular Ad11p.

In one embodiment the virus of the disclosure, such as a chimeric oncolytic virus has fibre, hexon and penton proteins from the same serotype, for example Ad11, in particular Ad11p, for example found at positions 30811-31788, 18254-21100 and 13682-15367 of the genomic sequence of the latter.

A virus of a distinct serotype to a first virus may be from the same subgroup or a different subgroup but will always be from a different serotype. In one embodiment the combinations are as follows in (first Ad serotype: second Ad serotype): AA, AB, AC, AD, AE, AF, AG, BB, BC, BD, BF, BG, CC, CD, CE, CF, CG, DD, DE, DF, DG, EE, EF, EG, FF, FG and GG.

In one embodiment the chimeric E2B region is derived from Ad3 and Ad11 (in particular Ad11p).

In one embodiment the E2B region is the sequence shown in SEQ ID NO: 2 herein.

Mammalian cells are cell derived from a mammal. In one embodiment the mammalian cells are selected from the group comprising HEK, CHO, COS-7, HeLa, Viro, A549, PerC6 and GMK, in particular HEK293.

In one embodiment the cells are grown in adherent or suspension culture, in particular a suspension culture.

Culturing mammalian cells as employed herein refers to the process where cells are grown under controlled conditions ex vivo. Suitable conditions are known to those in the art and may include temperatures such as 37° C. The CO₂ levels may need to be controlled, for example kept at a level of 5%. Details of the same are given in the text Culture of Animal Cells: A Manual of Basic Techniques and Specialised Applications Edition Six R. Ian Freshney, Basic Cell Culture (Practical Approach) Second Edition Edited by J. M. Davis.

Usually the cells will be cultured to generate sufficient numbers before infection with the adenovirus. These methods are known to those skilled in the art or are readily available in published protocols or the literature.

Generally the cells will be cultured on a commercial scale, for example 5 L, 10 L, 15 L, 20 L, 25 L, 30 L, 35 L, 40 L, 45 L, 50 L, 100 L, 200 L, 300 L, 400 L, 500 L, 600 L, 700 L, 800 L, 900, 1000 L or similar.

Media suitable for culturing mammalian cells include but are not limited to EX-CELL® media from Sigma-Aldrich, such as EX-CELL° 293 serum free medium for HEK293 cells, EX-CELL® ACF CHO media serum free media for CHO cells, EX-CELL® 302 serum free media for CHO cells, EX-CELL CD hydrolysate fusion media supplement, from Lonza RMPI (such as RMPI 1640 with HEPES and L-glutamine, RMPI 1640 with or without L-glutamine, and RMPI 1640 with UltraGlutamine), MEM and DMEM, SFMII medium.

In one embodiment the medium is serum free. This is advantageous because it facilitates registration of the manufacturing process with the regulatory authorities.

The viruses of the present disclosure, such as chimeric oncolytic viruses have different properties to those of adenoviruses used as vectors such as Ad5, this includes the fact that they can be recovered from the medium without the need for cell lysis. Thus, whilst not wishing to be bound by theory, the viruses appear to have mechanisms to exit the cell.

Furthermore, the viruses of the present disclosure, such as chimeric oncolytic adenoviruses do not seem to associate or adhere the cells after exiting the same, which also facilitates recovery from the supernatant, in particular when the cell culturing conditions are optimised.

In addition the chimeric oncolytic viruses do not appear to degrade, even when the culturing process is extended to 70 hours or more. The degradation of the virus can be checked by assaying the infectivity of the virus. The infectivity of the virus decreases as the viral particles degrade.

In one embodiment the culturing period is in the range 30 to 100 hours, for example 35 to 70 hours, for example 40, 45, 50, 55, 60 or 65 hours.

In one embodiment the culturing period is 65, 70, 75, 80, 85, 90, 95 hours or more.

In one embodiment over 90% of the chimeric oncolytic virus is present in the supernatant at the 64 hour timepoint, for example, 91, 92, 93, 94, 95, 96, 97, 98, 99 or 100%, such as 95% or more, particularly 98% or more.

In one embodiment significant amounts of virus are in media post 38 hours. For example, over 50%, particularly over 70% of the virus is in the media post 38 hours.

In one embodiment the maximum total virus production is achieved at about 40 to 60 hours post-infection, for example 49 hours post-infection. In one embodiment the decrease in virus production following the maximum is slow.

In one embodiment the maximum total virus production is achieved at about 70 to 90 hours post-infection.

Surprisingly, the present inventors have found that, when employing the process, the cells maintain high viability (such as 80 to 90% viability) post-infection for over 90 hours. Thus in one embodiment the harvesting and process may continue as long as the cells remain viable.

Maximum total virus production as employed herein means the total number of viral particles produced per cell and encompasses viral particles in the supernatant and the cell.

In one embodiment the virus production in the supernatant for ColoAd1 at 49 hours post-infection is about 20000 to 30000 viral particles per cell (vp/cell). For example 26000 vp/cell.

In one embodiment the virus production in the supernatant for NG135 at 49 hours post-infection is about 20000 to 30000 vp/cell, for example 26000 vp/cell.

In one embodiment the virus production for NG76 at 49 hours post-infection is about 6000 to 10000 vp/cell, for example 8000 vp/cell.

In one embodiment there is less than 10% detectable virus in the CVL pellet at the 64 hour timepoint, i.e. post 64 hours, such as 9, 8, 7, 6, 5, 4, 3, 2, 1% detectable virus. Example 6 describes how the CVL was obtained.

CVL as employed herein means the crude viral lysate.

Culturing cells may employ a perfusion culture, fed batch culture, batch culture, a steady state culture, a continuous culture or a combination of one or more of the same as technically appropriate, in particular a perfusion culture.

In one embodiment the process is a perfusion process, for example a continuous perfusion process.

In one embodiment the culture process comprises one or more media changes. This may be beneficial for optimising cell growth, yield or similar. Where a medium change is employed, it may be necessary to recover virus particle from the media being changed. These particles can be combined with the main virus batch to ensure the yield of virus is optimised. Similar techniques may also be employed with the medium of a perfusion process to optimise virus recovery.

In one embodiment the culture process does not include a medium change step. This may be advantageous because no viral particles will be lost and therefore yield may be optimised.

In one embodiment the culture process comprises one or more cell additions or changes. Cell addition change as employed herein refers to replenishing some or all of the cells and optionally removing dead cells.

In one embodiment the chimeric oncolytic adenovirus during culture is at concentration in the range 20 to 150 particles per cell (ppc), such as 40 to 100 ppc, in particular 50 ppc.

Lower values of virus concentrations, such as less than 100 ppc, in particular 50 ppc may be advantageous because this may result in increased cell viability compared to cultures with higher virus concentrations, particularly when cell viability is measured before harvesting.

Low cell viability can result in cell lysis which may expose the cell to enzymes, which with time may attack the virus. However, in a dynamic process such as cell culturing a percentage, such as a small percentage of cells may be unviable. This does not generally cause significant problems in practice.

In one embodiment cell viability is around 85 to 95% during the process, for example at the 96 hour timepoint (i.e. 96 hours post-infection) when infected with ColoAd1, such as 90% viability.

In one embodiment cell viability is around 80 to 90% during the process, for example at the 96 hour timepoint (i.e. 96 hours post-infection) when infected with NG76, such as 83% viability.

In one embodiment cell viability is around 80 to 90% during the process, for example at the 96 hour timepoint (i.e. 96 hours post-infection) when infected with NG135, such as 85% viability.

In one embodiment cell viability is around 80 to 90% during the process, for example at the 96 hour timepoint (i.e. 96 hours post-infection) when infected with Ad11. For example 85% viability.

In one embodiment the medium and/or cells are supplements or replenished periodically.

In one embodiment the cells are harvested during the process, for example at a discrete timepoint or timepoints or continuously.

In one embodiment of the process the mammalian cells are infected with a starting concentration of virus of 1-9×10⁴ vp/ml or greater, such as 1-9×10⁵, 1-9×10⁶, 1-9×10⁷, 1-9×10⁸, 1-9×10⁹, in particular 1-5×10⁶ vp/ml or 2.5-5×10⁸ vp/ml.

In one embodiment of the process the mammalian cells are infected at a starting concentration of 1×10⁶ cells/ml at about 1 to 200 ppc, for example 40 to 120 ppc, such as 50 ppc.

Ppc as employed herein refers to the number of viral particles per cell.

In one embodiment the process is run at about 35 to 39° C. For example 37° C.

In one embodiment the process run at about 4-6% CO₂. For example 5% CO₂.

In one embodiment the media containing the virus, such as the chimeric oncolytic viral particles is filtered to remove the cells and provide crude supernatant for further downstream processing.

In one embodiment a tangential flow filter is employed.

In one embodiment medium is filtered employing Millipore's Millistak+® POD system with cellulose based depth filters. Millistak+® depth filter medium is offered in a scalable, disposable format, the Pod Filter System. It is ideal for a wide variety of primary and secondary clarification applications, including cell cultures.

Millistak+® Pod filters are available in three distinct series of media grades in order to meet specific application needs. Millistak+® DE, CE and HC media deliver optimal performance through gradient density matrix as well as positive surface charge properties. In one embodiment the filtration is effected using tangential flow technology, for example employing the Cogent™ M system comprising a Pellicon Mini cassette membrane holder, pressure sensors, 10 litre recycle tank with mixer, retentate flow meter, weigh scale, feed pump, transfer pump, piping and valves. Control and operation of the system is manual with an exception of semi-automatic diafiltration/concentration. The operator has manual control of pump speeds, all valves and operational procedures. The virus can also, if desired, be formulated into the final buffer in this step.

Thus in one embodiment in the filtration step, concentrated and conditioned adenovirus material is provided in a final or near final formulation.

In one embodiment the process comprises two or more filtration steps.

In one embodiment the downstream processing comprises Millistak+POD system 35 CE and 50 CE cassettes followed by an opticap XL 10 express 0.5/0.2 um membrane filter in series.

In one embodiment the process further comprises a purification step, selected from a CsCl gradient, chromatography step such as size exclusion chromatography, ion-exchange chromatography in particular anion-exchange chromatography, and a combination thereof.

Ion exchange chromatography binds DNA very strongly and typically is the place were any residual DNA is removed. The ion exchange resin/membrane binds both the virus and the DNA and during salt gradient elusion the virus normally elutes off the column first (low salt gradient) and the DNA is eluted at a much higher salt concentration since the interaction of the DNA with the resin is stronger than the virus.

In one embodiment the chromatography step or steps employ monolith technology, for example available from BIA Separations.

In one embodiment Sartobind Q (quaternary amine membrane purification process) is employed as a purification step.

In one embodiment Source Q RESIN is employed in a purification step.

In one embodiment Sartobind Q is employed followed by Source Q RESIN in downstream processing of the isolated virus.

In one embodiment Source Q is employed in the purification step.

In one embodiment after purification the virus prepared contains less than 80 ng/mL of contaminating DNA, for example between 60 ng/mL and 10 ng/mL.

In one embodiment substantially all the contaminating DNA fragments are 700 base pairs or less, for example 500 bp or less, such as 200 bp or less.

In one embodiment the residual benzonase content in the purified virus product is 1 ng/mL or less, such as 0.5 ng/mL or less.

In one embodiment the residual host cell protein content in the purified virus product in 20 ng/mL or less, for example 15 ng/mL or less, in particular when measured by an ELISA assay.

In one embodiment the residual tween in the purified virus product is 0.1 mg/mL or less, such as 0.05 mg/mL or less.

In one embodiment the virus has a hexon and fibre from a group B adenovirus, for example Ad11 and in particular wherein the virus is ColoAd1.

In one embodiment there is provided isolated purified ColoAd1 wherein the contaminating DNA content is less than 80 ng/mL.

ColoAd1 is disclosed in WO 2005/118825 and the full sequence for the virus is provided herein, namely SEQ ID No: 1.

Alternative chimeric oncolytic viruses include OvAd1 and OvAd2, which are SEQ ID NO: 2 and 3 respectively disclosed in WO 2008/080003 and incorporated herein by reference.

In one embodiment the virus is replication competent. Replication competent virus as employed herein refers to a virus that is capable of replication without the assistance of a complementary cell line encoding an essential viral protein, such as that encoded by the E1 region (also referred to as a packaging cell line) and virus capable of replicating without the assistance of a helper virus.

In one embodiment the virus of the disclosure, such as the chimeric oncolytic virus of the present disclosure comprises one or more transgenes, for example one or more transgenes encoding therapeutic peptide(s) or protein sequence(s).

In one embodiment the chimeric oncolytic virus encodes at least one transgene. Suitable transgenes include so called suicide genes such as p53; polynucleotide sequences encoding cytokines such as IL-2, IL-6, IL-7, IL-12, IL-15, IL-18, IL-21, GM-CSF or G-CSF, an interferon (eg interferon I such as IFN-alpha or beta, interfon II such as IFN-gamma), a TNF (eg TNF-alpha or TNF-beta), TGF-beta, CD22, CD27, CD30, CD40, CD120; a polynucleotide encoding a monoclonal antibody, for example trastuzamab, cetuximab, panitumumab, pertuzumab, epratuzumab, an anti-EGF antibody, an anti-VEGF antibody and anti-PDGF antibody, an anti-FGF antibody.

A range of different types of transgenes, and combinations thereof, are envisaged that encode molecules that themselves act to modulate tumour or immune responses and act therapeutically, or are agents that directly or indirectly inhibit, activate or enhance the activity of such molecules. Such molecules include protein ligands or active binding fragments of ligands, antibodies (full length or fragments, such as Fv, ScFv, Fab, F(ab)′2 or smaller specific binding fragments), or other target-specific binding proteins or peptides (e.g. as may be selected by techniques such as phage display etc), natural or synthetic binding receptors, ligands or fragments, specific molecules regulating the transcription or translation of genes encoding the targets (e.g. siRNA or shRNA molecules, transcription factors). Molecules may be in the form of fusion proteins with other peptide sequences to enhance their activity, stability, specificity etc (e.g. ligands may be fused with immunoglobulin Fc regions to form dimers and enhance stability, fused to antibodies or antibody fragments having specificity to antigen presenting cells such as dendritic cells (e.g. anti-DEC-205, anti-mannose receptor, anti-dectin). Transgenes may also encode reporter genes that can be used, for example, for detection of cells infected with the “insert-bearing adenovirus”, imaging of tumours or draining lymphatics and lymph nodes etc.

In one embodiment the cancer cell infected with the chimeric oncolytic virus is lysed releasing the contents of the cell which may include the protein encoded by a transgene.

In one embodiment 40 to 93% or more of the total virus replicated in the cells is recoverable from the media, for example 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91 or 92% of the total virus is recoverable, such as 94, 95, 96, 97, 98, 99 or 100% of the total virus recoverable.

In one embodiment the process is a GMP manufacturing process, such as a cGMP manufacturing process.

In one embodiment the process further comprises the step formulating the virus in a buffer suitable for storage.

In one embodiment the present disclosure extends to virus or viral formulations obtained or obtainable from the present method.

Known methods for cell lysis include employing lysis buffer (pH 8.0) containing MgCl₂ and detergent, e.g. 1% Tween-20. Cell lysis is performed without pH or pO₂ controls. Rocking and heating are used. Lysis is continued for 1.5-2 hours.

Freeze-thawing multiple times is also a routine method of cell lysis.

Benzonase (Merck), 100 U/ml, is used to digest host cell DNA. Benzonase treatment is done for 30 min in +37° C. Benzonase is stopped with high salt incubation for 1 hour at RT.

Pulmozyme may also be employed in cell lysis.

Alternative methods for cell lysis include centrifuging cell suspension at 1000×g, 10 min at 4° C. Resuspending the cell pellet into 1 ml of Ex-Cell medium 5% glycerol and releasing the viruses from the cells by freeze-thaw by freezing tubes containing the responded cells from the pellet in liquid nitrogen for 3-5 minutes and thaw at +37° C. water bath until thawed. Generally the freeze and thaw step is repeated twice more. This cycle releases viruses from the cells. After the last thaw step remove the cell debris by centrifugation 1936×g, 20 min at +4° C.

In the context of the resent application, medium and media may be used interchangeably.

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The present invention is further described by way of illustration only in the following examples, which refer to the accompanying Figures, in which:

### EXAMPLES

### Example 1

Suspension HEK293s (1×10⁶ cells/mL in 125 mL shake flasks at 100 rpm) were infected at MOI 10 and fed with CD293 media 2 hours after infection with ColoAd1. Samples were taken 6, 24, 30, 48, 54 and 78 hours after infection. The supernatant was separated from the cells by centrifugation and the cell pellet resuspended in cell lysis buffer. The amount of infectious ColoAd1 particles in the cells and supernatant were determined by immunostaining infectivity assay and expressed as a proportion of the total at each time point. N=1, error bars (SD) show triplicate infections. Results are shown in FIG. 2.

### Example 2

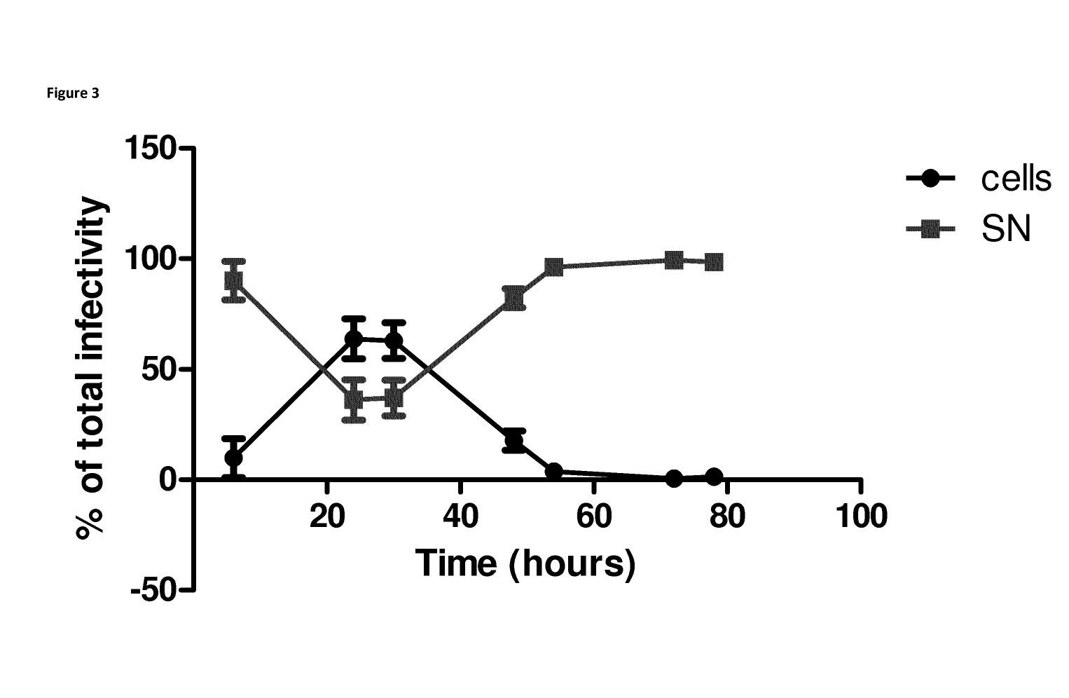

Adherent HEK293s (1×10⁶ cells/mL in 1 mL of 24-well plate) were infected at MOI 10 (in 2% FCS containing media). At 6, 24, 30, 48, 54, 72 and 78 hours after infection with ColoAd1 supernatant was removed, and cells detached from the surface before re-suspending in cell lysis buffer. The amount of infectious ColoAd1 particles associated with the cells and supernatant were determined by immunostaining infectivity assay and expressed as a proportion of the total at each time point. N=1, error bars (SD) show triplicate infections. Results shown in FIG. 3.

### Example 3

### ColoAd1 Cultured in a HEK 293 Suspension Culture

Infection conditions for oncolytic virus ColoAd1 were tested in a small scale suspension HEK293 culture. Cells were cultured for 96 hours prior to infection using Ex-Cell—6 mM L-glutamine—50 g/ml50 IU/ml Penicllin/Streptomycin at +37° C. and 5% CO₂.

Cell counting was performed using Bürker cell hemacytometer and Trypan Blue stain (Invitrogen, 15250-061). For larger dilutions (dilution factor 3 or greater) cell culture medium with Trypan blue was used. Two virus dilutions (50 and 100 particles per cell) and six harvesting time points (40, 43, 36, 49, 64, and 70 h) were tested. All the testing was done in duplicate shaker flasks. The viral particle concentrations of the samples were analysed with AEX-HPLC and the results are shown in Tables 3 and 4.

TABLE 3
AEX-HPLC results of the cell samples (intracellular virus concentrations)
of suspension HEK239 culture in infection condition testing.

| | | AEX-HPLC | Vol- | | | |
|---|---|---|---|---|---|---|
| | Infection | titer | ume | | Average | Produced |
| ppc | time (h) | (vp/ml) | (ml) | Total vp | total vp | vp/cell |
| 50 | 40 | 6.99E+11 | 1.05 | 7.34E+11 | 1.12E+12 | 70914 |
| | | 1.25E+12 | 1.20 | 1.50E+12 | | |
| | 43 | 6.89E+11 | 1.05 | 7.24E+11 | 6.52E+11 | 89169 |
| | | 5.05E+11 | 1.15 | 5.81E+11 | | |
| | 46 | 5.69E+11 | 1.20 | 6.82E+11 | 6.39E+11 | 99401 |
| | | 4.96E+11 | 1.20 | 5.96E+11 | | |
| | 49 | 7.43E+11 | 1.15 | 8.55E+11 | 8.77E+11 | 155253 |
| | | 7.50E+11 | 1.20 | 9.00E+11 | | |
| | 64 | 6.18E+11 | 1.20 | 7.42E+11 | 7.25E+11 | 228787 |
| | | 5.89E+11 | 1.20 | 7.07E+11 | | |
| | 70 | 6.28E+11 | 1.20 | 7.54E+11 | 8.34E+11 | 262976 |
| | | 7.62E+11 | 1.20 | 9.14E+11 | | |
| 100 | 40 | 6.37E+11 | 1.20 | 7.64E+11 | 4.57E+11 | 60914 |
| | | 1.25E+11 | 1.20 | 1.50E+11 | | |
| | 43 | 8.16E+11 | 1.10 | 8.97E+11 | 9.10E+11 | 107440 |
| | | 9.23E+11 | 1.00 | 9.23E+11 | | |
| | 46 | 5.77E+11 | 1.20 | 6.92E+11 | 7.20E+11 | 155171 |
| | | 6.23E+11 | 1.20 | 7.48E+11 | | |
| | 49 | 7.38E+11 | 1.20 | 8.85E+11 | 8.65E+11 | 215871 |
| | | 7.05E+11 | 1.20 | 8.45E+11 | | |
| | 64 | 7.06E+11 | 1.10 | 7.76E+11 | 8.35E+11 | 321975 |
| | | 7.45E+11 | 1.20 | 8.94E+11 | | |
| | 70 | 6.69E+11 | 1.20 | 8.03E+11 | 7.82E+11 | 351378 |
| | | 5.85E+11 | 1.30 | 7.60E+11 | | |

TABLE 4
AEX-HPLC results of the medium samples and total
intracellular and extracellular virus amount
of suspension HEK239 culture in infection condition testing.

| | Infec- | AEX- | | | | Total | |
|---|---|---|---|---|---|---|---|
| | tion | HPLC | Vol- | | | vp in | % |
| | time | titer | ume | | Average | cells + in | in |
| ppc | (h) | (vp/ml) | (ml) | Total vp | total vp | medium | medium |
| 50 | 40 | 3.84E+10 | 28.0 | 1.08E+12 | 1.01E+12 | 2.13E+12 | 47 |
| | | 3.42E+10 | 27.5 | 9.42E+12 | | | |
| | 43 | 7.23E+10 | 27.5 | 1.99E+12 | 2.02E+12 | 2.68E+12 | 76 |
| | | 7.22E+10 | 28.5 | 2.06E+12 | | | |
| | 46 | 1.06E+11 | 27.5 | 2.93E+12 | 2.34E+12 | 2.98E+12 | 79 |
| | | 6.21E+10 | 28.3 | 1.76E+12 | | | |
| | 49 | 1.33E+11 | 27.5 | 3.67E+12 | 3.78E+12 | 4.66E+12 | 81 |
| | | 1.37E+11 | 28.3 | 3.89E+12 | | | |
| | 64 | 2.18E+11 | 27.3 | 5.95E+12 | 6.13E+12 | 6.86E+12 | 89 |
| | | 2.24E+11 | 28.3 | 6.33E+12 | | | |
| | 70 | 2.57E+11 | 27.3 | 7.03E+12 | 7.05E+12 | 7.89E+12 | 89 |
| | | 2.52E+11 | 28.1 | 7.09E+12 | | | |
| 100 | 40 | 4.63E+10 | 28.0 | 1.30E+12 | 1.37E+12 | 1.83E+12 | 75 |
| | | 5.25E+10 | 27.5 | 1.44E+12 | | | |
| | 43 | 7.44E+10 | 27.5 | 2.05E+12 | 2.31E+12 | 3.22E+12 | 72 |
| | | 9.05E+10 | 28.5 | 2.58E+12 | | | |
| | 46 | 1.37E+11 | 27.7 | 3.79E+12 | 3.94E+12 | 4.66E+12 | 85 |
| | | 1.42E+11 | 28.7 | 4.08E+12 | | | |
| | 49 | 1.92E+11 | 27.9 | 5.36E+12 | 5.61E+12 | 6.48E+12 | 87 |
| | | 2.06E+11 | 28.5 | 5.86E+12 | | | |
| | 64 | 3.12E+11 | 27.4 | 8.55E+12 | 8.82E+12 | 9.66E+12 | 91 |
| | | 3.21E+11 | 28.3 | 9.10E+12 | | | |
| | 70 | 3.49E+11 | 26.9 | 9.40E+12 | 9.76E+12 | 1.05E+13 | 93 |
| | | 3.55E+11 | 28.5 | 1.01E+13 | | | |

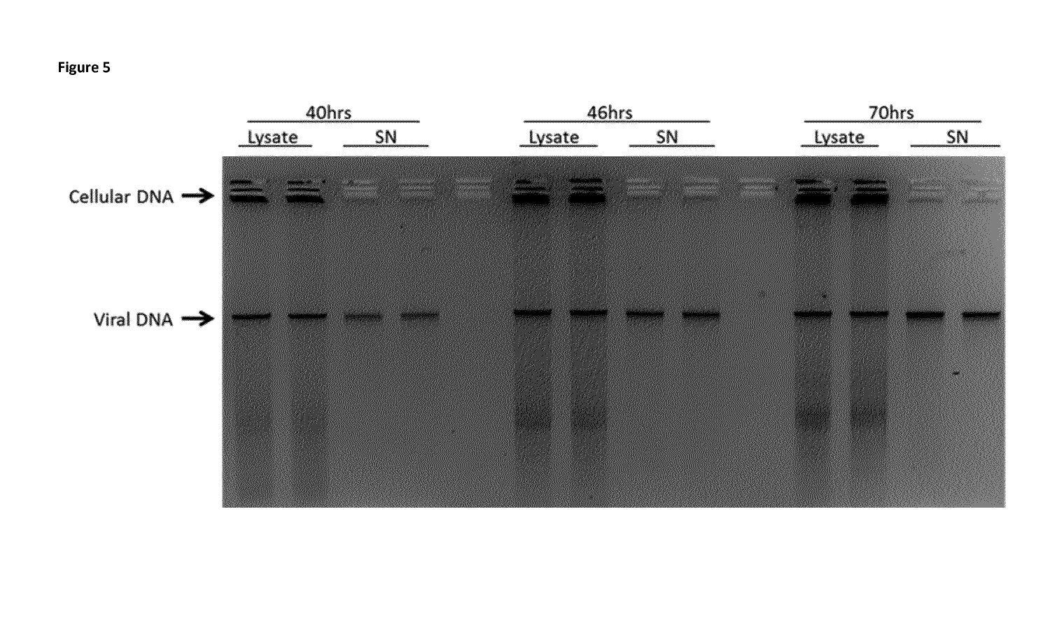

The highest amount of viral particles was produced when infecting the cells for 70 hours with 100 ppc (the average results of duplicate flasks 1.05E+13vp, Table 4). At that time point, 93% of the viral particles were in the medium. It can be seen from FIG. 5, that the total amount of virus increased up to 70 hours, but the curve seemed to start approaching plateu after 64 hours. Already at 43 hours, over half of the virus is in the culture medium, however, in the suspension production process, also the viral particles in the medium can be captured during purification. In 70 hours, MOI of 100 ppc produced 2.6E+12 more viral particles than 50 ppc (and 2.8E+12 more in 64 h). But even with 50 ppc, the production capacity of the cells appeared to be close to the maximum: the intracellular virus amount remained fairly constant during the time range of 40-70 hours.

### Example 4

### Example of ColoAd1 Cultured in Adherent HEK 293 Cells

Adherent HEK293 cells were seeded at 4.8×10₆ cells per flask in 185 cm₂ cell culture flasks (24 pieces) 72 hours prior to infection. Cell culturing was performed using DMEM—10% FBS—2 mM L-glutamine at +37° C. and 5% CO₂. Cell number was counted from one cell culture flask on the day of infection resulting 40.6×10⁶ cells/flask. The tested particles per cell (ppc) were 200, 100 and 50. After infection the cells may be cultured for between 35 to 70 hours.

### Example 5

### Visualisation of Cellular and Viral DNA in the Cell Lysate (Lysate) or Supernatant (SN) of ColoAd1 Infected HEK293 Cells at 40 Hrs, 46 Hrs and 70 Hrs Post-Infection

HEK293 cells infected with ColoAd1 at 50 particles per cell were harvested 40 hours, 46 hours or 70 hours post-infection. The culture supernatants and the cell lysates were collected and total DNA extracted. Equivalent volumes of purified lysate or supernatant DNA were loaded in duplicate onto a 0.7% agarose gel and the DNA was separated electrophoresis. Significant cellular DNA could be detected at the top of the gel and as a smear in all lanes containing DNA extracted from cell lysates, however only very low levels of cellular DNA could be detected in lanes containing DNA extracted from supernatant (SN). In contrast, viral DNA could be detected in all samples and the total detectable viral DNA observably increased in the supernatant over time. Results are shown in FIG. 5.

### Example 6

ColoAd1, NG-135, NG-76, Ad5 and Ad11p (referred to in the figures and tables as Ad11) were compared for the relative levels of expression of virus particles associated with the cell pellet (CVL) or in the supernatant.

TABLE 5
Total adenoviral particle concentration of virus (by HPLC assay).

| | AEX HPLC | 1:10 | 1:100 |
|---|---|---|---|
| Viruses used | titer vp/ml | Dilution | Dilution |
| ColoAd 1 (CA111001) | 3.00E+12 | 3.00E+11 | 3.00E+10 |
| NG 135 Banded(full Ab) | 2.69E+12 | 2.69E+11 | 2.69E+10 |
| NG 76-03 Banded(ScFv) | 6.91E+11 | 6.91E+10 | 6.91E+09 |
| Ad5 Banded | 1.59E+12 | 1.59E+11 | 1.59E+10 |
| Ad11 Banded | 2.30E+11 | 2.30E+10 | 2.30E+09 |

Suspension HEK293 cells (293f) were cultured in duplicate shaker flasks containing 40 ml working volume of SFMII media supplemented with 4 mM L-glutamine and 50 μg/ml/50 IU/ml

Penicillin/Streptomycin and infected at 10⁶ cells/ml with viruses at a ratio of 50 virus particles per cell (ppc).

The cell expansion was started by thawing one vial of cells and continued cell expansion for 3 weeks until a total of 4.8×10⁸ cells required for this study was achieved. Three days before infection, the HEK 293 suspension cells were seeded in a single one litre shaker flask using 4×10⁵ cells/ml in 428 ml of SFMII medium per flask (3.4×10⁸ cells/flask) and incubated in a shaker incubator at +37° C., 5% CO₂ &115 rpm.

On the day of infection the cell count was performed and based on cell density, 225 ml of cell suspension at 2.15×10⁶ cell/ml was used for the study. Remaining cells were discarded. HEK 293 suspension cells were infected with one of the four different viruses (see Table 5) at 50 ppc in duplicate.

A 1:100 dilution of each virus was performed in SFMII growth medium prior to infection of cells (for virus concentration refer to Table 5).

TABLE 6
Infection calculation and dilution of virus prior to infection

| | | Vol- | | | total | | virus sol | |
|---|---|---|---|---|---|---|---|---|
| | | ume | total | | vp | Diluted | required | |
| Virus | cells/ml | (ml) | cells | ppc | needed | vp/ml | (ml) | Notes |
| ColoAd1 | 1.00E+06 | 40 | 4.00E+07 | 50 | 2.00E+09 | 3.00E+10 | 0.067 | Total 2 × 67 ul of the |
| | | | | | | | | 1:100 (10 + 990) |
| | | | | | | | | used for infection of 2 flasks |
| NG 135 | 1.00E+06 | 40 | 4.00E+07 | 50 | 2.00E+09 | 2.69E+10 | 0.074 | Total 2 × 74 ul of the |
| | | | | | | | | 1:100 (10 + 990) |
| | | | | | | | | used for infection of 2 flasks |
| NG 76-03 | 1.00E+06 | 40 | 4.00E+07 | 50 | 2.00E+09 | 6.91E+09 | 0.290 | Total 2 × 291 ul of the |
| | | | | | | | | 1:100 (10 + 990) |
| | | | | | | | | used for infection of 2 flasks |
| Ad5 | 1.00E+06 | 40 | 4.00E+07 | 50 | 2.00E+09 | 1.59E+10 | 0.126 | Total 2 × 126 ul of |
| | | | | | | | | the 1:100 (10 + 990) |
| | | | | | | | | used for infection of 2 flasks |
| Ad11 | 1.00E+05 | 40 | 4.00E+07 | 50 | 2.40E+09 | 2.30E+09 | 0.870 | Total 2 × 870 ul of the |
| | | | | | | | | 1:100 (10 + 990) |
| | | | | | | | | used for infection of 2 flasks |

Infection of suspension HEK293 cells with virus diluted as follows:

225 ml of cell suspension at 2.15×10⁶ was centrifuged and the cell pellets was resuspended in 480 ml media to adjust the cell concentration to 1×10⁶ cells/ml,

Thereafter, 10 shaker flasks with working volume of 40 ml per shaker flask were prepared. Dual flask were labelled as ColoAd1 -A1A2, NG 135-B1B2, NG 76-C1C2, Ad5-D1D2 and Ad11E1E2 respectively.

All labelled flasks were infected with 50 ppc virus in accordance with Table 6.

All shaker flasks were placed in a shaking incubator at +37° C., 5% CO₂ &120 rpm until harvested.

At 40, 46, 49, 64, 70, 73 and 89 hours post infection, 2.5 ml samples were taken from each flask and the duplicates pooled to provide 5.0 ml samples. At 96 hours post-infection, all the cells were harvested. Cell viabilities were assessed using 0.5 ml and the remaining 4.5 ml volume was then centrifuged and the virus distribution between the supernatant and cell pellet for each virus was determined in the following way:

The cells were centrifuged at 1000×g, 10 min at 4° C.
After centrifugation, the supernatant was gently poured into a sterile container and 0.5 ml of 50% glycerol was added to the sample 1 ml aliquots were stored at −80° C. until analysis.
The cell pellet was suspended in 1 ml of SFMII medium containing 5% glycerol,

Intracellular virus was released from the cells by freeze-thaw as follows:
The centrifuge tubes were frozen in liquid nitrogen for 3-5 minutes and then transferred to a water bath set at +37° C. until thawed.
The freeze and thaw process was repeated twice more as described in step a, above.
After the final thaw step, the cell debris is removed from the crude viral lysate (CVL) by centrifugation at 1936×g for 20 min at +4° C. and transfer of the supernatant (CVL) to a new container.
The CVL was aliquoted as 100 μl samples and stored at −80° C. until analysis.

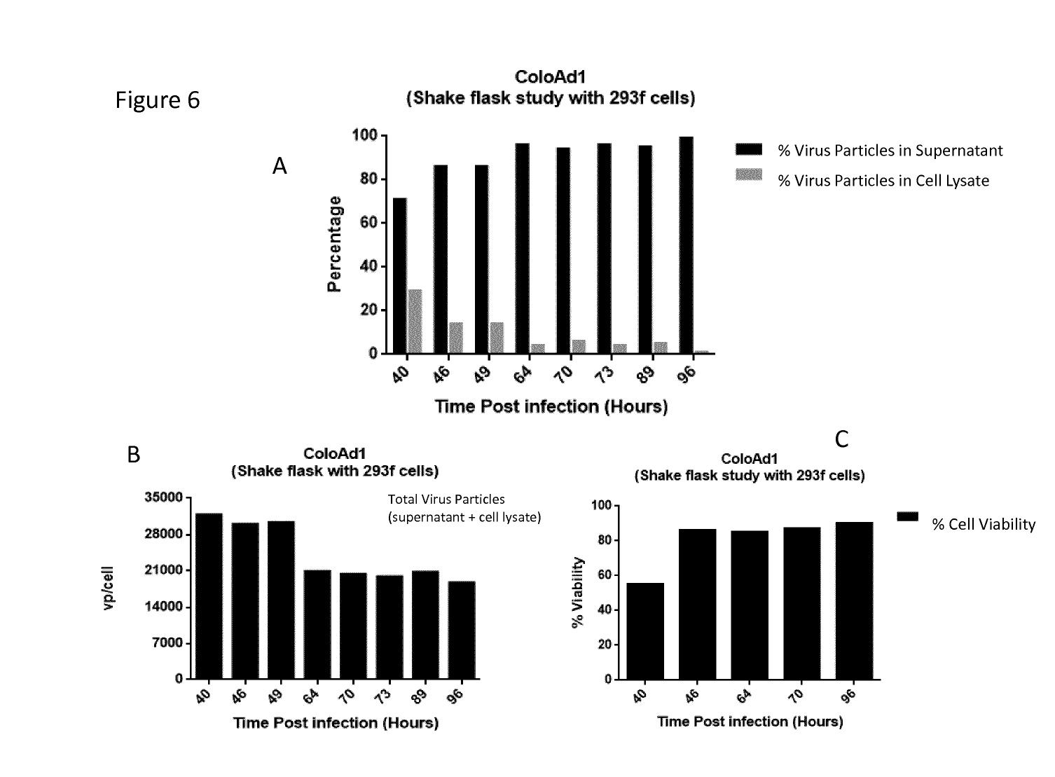

Total viral particle concentration (vp) from Crude Viral Lysate (CVL) and supernatant (SN) samples were analysed by AEX-HPLC assay. These values were then used to calculate the total number of virus particles per culture and the percentage in the SN and CVL for each sample time point. These are represented as bar graphs, together with the viability of the HEK293 cells, for ColoAd1, NG-135, NG-76, Ad5 and A11p in FIGS. 6-10 respectively. For ColoAd1, NG-135,NG-76 and Ad11p the majority of virus was in the culture supernatant whereas for Ad5 virus was entirely in the cell lysate (CVL), being undetectable in the supernatant. For all cultures, the viability of the HEK293 cells remained high over the 96 hours of culture.

For ColoAd1 (FIG. 6A), 84% was present in the supernatant at 40 hrs timepoint, and 98% present in the supernatant at 64 h timepoint with no detectable virus in the CVL (pellet) sample.

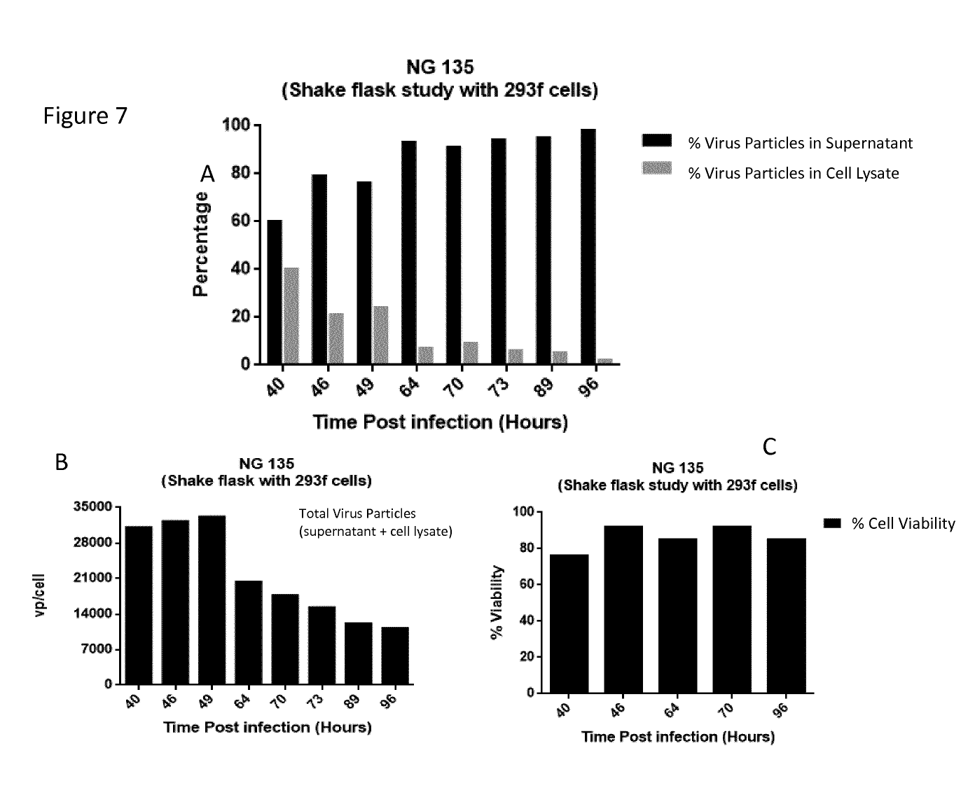

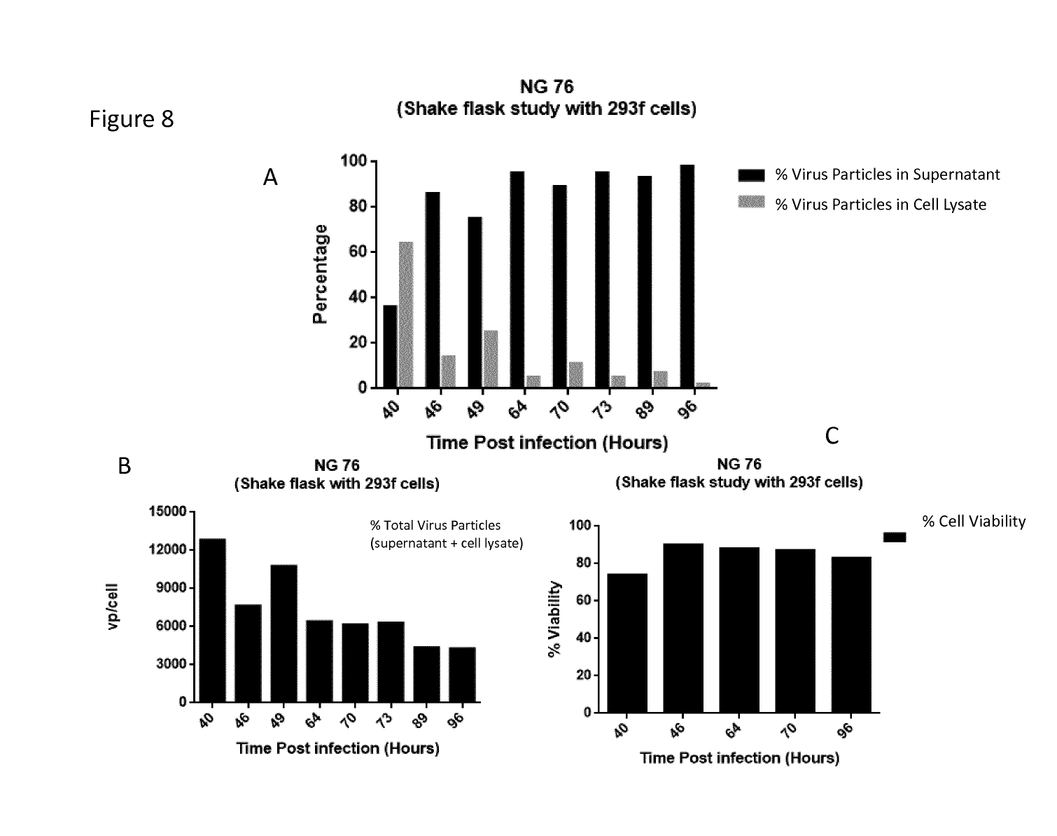

For Next Gen 135 (FIG. 7A) and Next Gen 76 (FIG. 8A) the trend of virus distribution is similar to ColoAd1. For NG135, 77% of virus was present in the supernatant at 40 hrs timepoint. This increased to 97% virus present in the the supernatant at 64 h timepoint. For the same time points, 56% and 98% of the virus for Next Gen 76 was present in the supernatant.

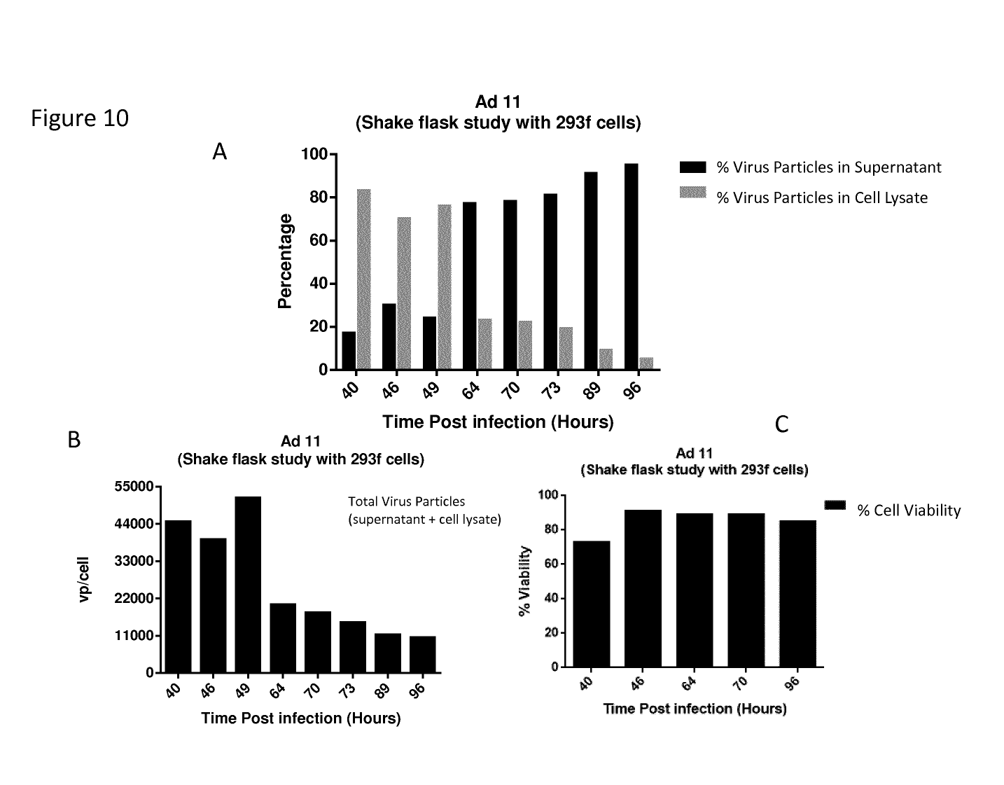

For Ad11 (FIG. 10A), 31% of the virus was present in the supernatant at 40 hrs timepoint, 88% present in the supernatant at 64 h timepoint and 98% of the virus present in the supernatant at 96 h timepoint, respectively.

For Ad5 (FIG. 9A), 100% of the virus was detected in CVL (pellet) sample with no virus detected in the supernatant.

For all viruses assessed in this study, the maximum level of virus was observed at 49 hrs post infection and there was a slow decrease in virus level observed thereafter in subsequent timepoints

For ColoAd1 (FIG. 6B) and NG135 (FIG. 7B) maximum virus level (27000 vp/cell) was observed at 49 hrs timepoint, while in NG76 (FIG. 8B) the maximum virus level (9200 vp/cell) was observed at same timepoint.

For Ad5 (FIG. 9B) the maximum virus level(11000 vp/cell) was observed at 89 hrs timepoint while in Ad11p (FIG. 10B) the maximum virus level (30000 vp/cell) was observed at same timepoint.

The total viral particle concentrations of all the supernatant and CVL samples analysed with AEX-HPLC are shown in Tables 7 and 8 and in FIGS. 6-10.

TABLE 7
AEX-HPLC assay results of ColoAd1, NG135 and NG76

| | | | AEX- | | AEX- | | | | |
|---|---|---|---|---|---|---|---|---|---|
| | | | HPLC | | HPLC | Vol- | | | |
| Virus | Infection | Sample | titer | | titer | ume | Total | Avg | Total |
| used | time (h) | Detail | vp/ml | DF | vp/ml | (ml) | vp | cells/ml | cells |
| ColoAd1 | 40 | SN 40 h ColoAd1 | 2.03E+10 | 1.11 | 2.25E+10 | 40.00 | 9.00E+11 | 1.00E+06 | 4.00E+07 |
| | | A1A2 | | | | | | | |
| | 46 | SN 46 h ColoAd1 | 2.31E+10 | 1.11 | 2.57E+10 | 37.50 | 9.63E+11 | 1.00E+06 | 3.75E+07 |
| | | A1A2 | | | | | | | |
| | 49 | SN 49 h ColoAd1 | 2.34E+10 | 1.11 | 2.60E+10 | 35.00 | 9.11E+11 | 1.00E+06 | 3.50E+07 |
| | | A1A2 | | | | | | | |
| | 64 | SN 64 h ColoAd1 | 1.81E+10 | 1.11 | 2.01E+10 | 32.50 | 6.52E+11 | 1.00E+06 | 3.25E+07 |
| | | A1A2 | | | | | | | |
| | 70 | SN 70 h ColoAd1 | 1.73E+10 | 1.11 | 1.92E+10 | 30.00 | 5.77E+11 | 1.00E+06 | 3.00E+07 |
| | | A1A2 | | | | | | | |
| | 73 | SN 73 h ColoAd1 | 1.71E+10 | 1.11 | 1.90E+10 | 27.50 | 5.22E+11 | 1.00E+06 | 2.75E+07 |
| | | A1A2 | | | | | | | |
| | 89 | SN 89 h ColoAd1 | 1.77E+10 | 1.11 | 1.97E+10 | 25.00 | 4.92E+11 | 1.00E+06 | 2.50E+07 |
| | | A1A2 | | | | | | | |
| | 96 | SN 96 h ColoAd1 | 1.84E+10 | 1.00 | 1.84E+10 | 22.50 | 4.15E+11 | 1.00E+06 | 2.25E+07 |
| | | A1A2 | | | | | | | |
| | 40 | CVL 40 h ColoAd1 | 2.10E+10 | 0.44 | 9.34E+09 | 40.00 | 3.74E+11 | 1.00E+06 | 4.00E+07 |
| | | A1A2 | | | | | | | |
| | 46 | CVL 46 h ColoAd1 | 9.69E+09 | 0.44 | 4.31E+09 | 37.50 | 1.61E+11 | 1.00E+06 | 3.75E+07 |
| | | A1A2 | | | | | | | |
| | 49 | CVL 49 h ColoAd1 | 9.75E+09 | 0.44 | 4.33E+09 | 35.00 | 1.52E+11 | 1.00E+06 | 3.50E+07 |
| | | A1A2 | | | | | | | |
| | 64 | CVL 64 h ColoAd1 | 1.87E+09 | 0.44 | 8.30E+08 | 32.50 | 2.70E+10 | 1.00E+06 | 3.25E+07 |
| | | A1A2 | | | | | | | |
| | 70 | CVL 70 h ColoAd1 | 2.54E+09 | 0.44 | 1.13E+09 | 30.00 | 3.39E+10 | 1.00E+06 | 3.00E+07 |
| | | A1A2 | | | | | | | |
| | 73 | CVL 73 h ColoAd1 | 1.98E+09 | 0.44 | 8.80E+08 | 27.50 | 2.42E+10 | 1.00E+06 | 2.75E+07 |
| | | A1A2 | | | | | | | |
| | 89 | CVL 89 h ColoAd1 | 2.43E+09 | 0.44 | 1.08E+09 | 25.00 | 2.70E+10 | 1.00E+06 | 2.50E+07 |
| | | A1A2 | | | | | | | |
| | 96 | CVL 96 h ColoAd1 | 6.10E+09 | 0.04 | 2.71E+08 | 22.50 | 6.10E+09 | 1.00E+06 | 2.25E+07 |
| | | A1A2 | | | | | | | |
| | | | | | Total | | | | Produced |
| | | | | | vp | Total | Total | | vp/cell |
| | | Cell | Total | Total | (SN | vp | vp | Produced | (SN |
| Virus | Produced | viability | vp | vp | + | (% in | (% in | vp/cell | + |
| used | vp/cell | % | (SN) | (CVL) | CVL) | SN) | CVL) | (in SN) | CVL) |
| ColoAd1 | 22492 | 55 | 9.00E+11 | 3.74E+11 | 1.27E+12 | 71 | 29 | 22492 | 31830 |
| | 25671 | 86 | 9.63E+11 | 1.61E+11 | 1.12E+12 | 86 | 14 | 25671 | 29977 |
| | 26019 | NA | 9.11E+11 | 1.52E+11 | 1.06E+12 | 86 | 14 | 26019 | 30350 |
| | 20058 | 85 | 6.52E+11 | 2.70E+10 | 6.79E+11 | 96 | 4 | 20058 | 20888 |
| | 19238 | 87 | 5.77E+11 | 3.39E+10 | 6.11E+11 | 94 | 6 | 19238 | 20366 |
| | 18989 | NA | 5.22E+11 | 2.42E+10 | 5.46E+11 | 96 | 4 | 18989 | 19869 |
| | 19660 | NA | 4.92E+11 | 2.70E+10 | 5.18E+11 | 95 | 5 | 19660 | 20739 |
| | 18428 | 90 | 4.15E+11 | 6.10E+09 | 4.21E+11 | 99 | 1 | 18428 | 18699 |
| | 9339 | | | | | | | | |
| | 4306 | | | | | | | | |
| | 4331 | | | | | | | | |
| | 830 | | | | | | | | |
| | 1129 | | | | | | | | |
| | 880 | | | | | | | | |
| | 1079 | | | | | | | | |
| | 271 | | | | | | | | |
| | | | AEX- | | AEX- | | | | |
| | | | HPLC | | HPLC | Vol- | | | |
| Virus | Infection | Sample | titer | | titer | ume | Total | Avg | Total |
| used | time (h) | Detail | vp/ml | DF | vp/ml | (ml) | vp | cells/ml | cells |
| NG-135 | 40 | SN 40 h NG135 B1B2 | 1.66E+10 | 1.11 | 1.85E+10 | 40.00 | 7.39E+11 | 1.00E+06 | 4.00E+07 |
| | 46 | SN 46 h NG135 B1B2 | 2.31E+10 | 1.11 | 2.56E+10 | 37.50 | 9.60E+11 | 1.00E+06 | 3.75E+07 |
| | 49 | SN 49h NG135 B1B2 | 2.26E+10 | 1.11 | 2.51E+10 | 35.00 | 8.78E+11 | 1.00E+06 | 3.50E+07 |
| | 64 | SN 64 h NG135 B1B2 | 1.70E+10 | 1.11 | 1.88E+10 | 32.50 | 6.12E+11 | 1.00E+06 | 3.25E+07 |
| | 70 | SN 70 h NG135 B1B2 | 1.44E+10 | 1.11 | 1.60E+10 | 30.00 | 4.80E+11 | 1.00E+06 | 3.00E+07 |
| | 73 | SN 73 h NG135 B1B2 | 1.29E+10 | 1.11 | 1.44E+10 | 27.50 | 3.95E+11 | 1.00E+06 | 2.75E+07 |
| | 89 | SN 89 h NG135 B1B2 | 1.03E+10 | 1.11 | 1.15E+10 | 25.00 | 2.87E+11 | 1.00E+06 | 2.50E+07 |
| | 96 | SN 96 h NG135 B1B2 | 1.10E+10 | 1.00 | 1.10E+10 | 22.50 | 2.46E+11 | 1.00E+06 | 2.25E+07 |
| | 40 | CVL 40 h NG135 | 2.82E+10 | 0.44 | 1.25E+10 | 40.00 | 5.02E+11 | 1.00E+06 | 4.00E+07 |
| | | B1B2 | | | | | | | |
| | 46 | CVL 46 h NG135 | 1.49E+10 | 0.44 | 6.63E+09 | 37.50 | 2.49E+11 | 1.00E+06 | 3.75E+07 |
| | | B1B2 | | | | | | | |
| | 49 | CVL 49 h NG135 | 1.81E+10 | 0.44 | 8.03E+09 | 35.00 | 2.81E+11 | 1.00E+06 | 3.50E+07 |
| | | B1B2 | | | | | | | |
| | 64 | CVL 64 h NG135 | 3.32E+09 | 0.44 | 1.48E+09 | 32.50 | 4.80E+10 | 1.00E+06 | 3.25E+07 |
| | | B1B2 | | | | | | | |
| | 70 | CVL 70 h NG135 | 3.73E+09 | 0.44 | 1.66E+09 | 30.00 | 4.97E+10 | 1.00E+06 | 3.00E+07 |
| | | B1B2 | | | | | | | |
| | 73 | CVL 73 h NG135 | 2.09E+09 | 0.44 | 9.30E+08 | 27.50 | 2.56E+10 | 1.00E+06 | 2.75E+07 |
| | | B1B2 | | | | | | | |
| | 89 | CVL 89 h NG135 | 1.42E+09 | 0.44 | 6.31E+08 | 25.00 | 1.58E+10 | 1.00E+06 | 2.50E+07 |
| | | B1B2 | | | | | | | |
| | 96 | CVL 96 h NG135 | 5.31E+09 | 0.04 | 2.36E+08 | 22.50 | 5.31E+09 | 1.00E+06 | 2.25E+07 |
| | | B1B2 | | | | | | | |
| | | | | | Total | | | | Produced |
| | | | | | vp | Total | Total | | vp/cell |
| | | Cell | Total | Total | (SN | vp | vp | Produced | (SN |
| Virus | Produced | viability | vp | vp | + | (% in | (% in | vp/cell | + |
| used | vp/cell | % | (SN) | (CVL) | CVL) | SN) | CVL) | (in SN) | CVL) |
| NG-135 | 18468 | 76 | 7.39E+11 | 5.02E+11 | 1.24E+12 | 60 | 40 | 18468 | 31009 |
| | 25597 | 92 | 9.60E+11 | 2.49E+11 | 1.21E+12 | 79 | 21 | 25597 | 32230 |
| | 25075 | NA | 8.78E+11 | 2.81E+11 | 1.16E+12 | 76 | 24 | 25075 | 33106 |
| | 18840 | 85 | 6.12E+11 | 4.80E+10 | 6.60E+11 | 93 | 7 | 18840 | 20317 |
| | 15984 | 92 | 4.80E+11 | 4.97E+10 | 5.29E+11 | 91 | 9 | 15984 | 17640 |
| | 14369 | NA | 3.95E+11 | 2.56E+10 | 4.21E+11 | 94 | 6 | 14369 | 15299 |
| | 11463 | NA | 2.87E+11 | 1.58E+10 | 3.02E+11 | 95 | 5 | 11463 | 12094 |
| | 10954 | 85 | 2.46E+11 | 5.31E+09 | 2.52E+11 | 98 | 2 | 10954 | 11190 |
| | 12541 | | | | | | | | |
| | 6633 | | | | | | | | |
| | 8031 | | | | | | | | |
| | 1477 | | | | | | | | |
| | 1656 | | | | | | | | |
| | 930 | | | | | | | | |
| | 631 | | | | | | | | |
| | 236 | | | | | | | | |
| | | | AEX- | | AEX- | | | | |
| | | | HPLC | | HPLC | Vol- | | | |
| Virus | Infection | Sample | titer | | titer | ume | Total | Avg | Total |
| used | time (h) | Detail | vp/ml | DF | vp/ml | (ml) | vp | cells/ml | cells |
| NG-76 | 40 | SN 40 h NG76 C1C2 | 4.20E+09 | 1.11 | 4.66E+09 | 40.00 | 1.86E+11 | 1.00E+06 | 4.00E+07 |
| | 46 | SN 46 h NG76 C1C2 | 5.90E+09 | 1.11 | 6.54E+09 | 37.50 | 2.45E+11 | 1.00E+06 | 3.75E+07 |
| | 49 | SN 49 h NG76 C1C2 | 7.24E+09 | 1.11 | 8.04E+09 | 35.00 | 2.81E+11 | 1.00E+06 | 3.50E+07 |
| | 64 | SN 64 h NG76 C1C2 | 5.48E+09 | 1.11 | 6.09E+09 | 32.50 | 1.98E+11 | 1.00E+06 | 3.25E+07 |
| | 70 | SN 70 h NG76 C1C2 | 4.93E+09 | 1.11 | 5.48E+09 | 30.00 | 1.64E+11 | 1.00E+06 | 3.00E+07 |
| | 73 | SN 73 h NG76 C1C2 | 5.40E+09 | 1.11 | 6.00E+09 | 27.50 | 1.65E+11 | 1.00E+06 | 2.75E+07 |
| | 89 | SN 89 h NG76 C1C2 | 3.64E+09 | 1.11 | 4.04E+09 | 25.00 | 1.01E+11 | 1.00E+06 | 2.50E+07 |
| | 96 | SN 96 h NG76 C1C2 | 4.20E+09 | 1.00 | 4.20E+09 | 22.50 | 9.44E+10 | 1.00E+06 | 2.25E+07 |
| | 40 | CVL 40 h NG76 C1C2 | 1.83E+10 | 0.44 | 8.15E+09 | 40.00 | 3.26E+11 | 1.00E+06 | 4.00E+07 |
| | 46 | CVL 46 h NG76 C1C2 | 2.45E+09 | 0.44 | 1.09E+09 | 37.50 | 4.08E+10 | 1.00E+06 | 3.75E+07 |
| | 49 | CVL 49 h NG76 C1C2 | 6.12E+09 | 0.44 | 2.72E+09 | 35.00 | 9.52E+10 | 1.00E+06 | 3.50E+07 |
| | 64 | CVL 64 h NG76 C1C2 | 7.04E+08 | 0.44 | 3.13E+08 | 32.50 | 1.02E+10 | 1.00E+06 | 3.25E+07 |
| | 70 | CVL 70 h NG76 C1C2 | 1.52E+09 | 0.44 | 6.74E+08 | 30.00 | 2.02E+10 | 1.00E+06 | 3.00E+07 |
| | 73 | CVL 73 h NG76 C1C2 | 7.04E+08 | 0.44 | 3.13E+08 | 27.50 | 8.61E+09 | 1.00E+06 | 2.75E+07 |
| | 89 | CVL 89 h NG76 C1C2 | 7.04E+08 | 0.44 | 3.13E+08 | 25.00 | 7.83E+09 | 1.00E+06 | 2.50E+07 |
| | 96 | CVL 96 h NG76 C1C2 | 1.73E+09 | 0.04 | 7.71E+07 | 22.50 | 1.73E+09 | 1.00E+06 | 2.25E+07 |
| | | | | | Total | | | | Produced |
| | | | | | vp | Total | Total | | vp/cell |
| | | Cell | Total | Total | (SN | vp | vp | Produced | (SN |
| Virus | Produced | viability | vp | vp | + | (% in | (% in | vp/cell | + |
| used | vp/cell | % | (SN) | (CVL) | CVL) | SN) | CVL) | (in SN) | CVL) |
| NG-76 | 4657 | 74 | 1.86E+11 | 3.26E+11 | 5.12E+11 | 36 | 64 | 4657 | 12807 |
| | 6545 | 90 | 2.45E+11 | 4.08E+10 | 2.86E+11 | 86 | 14 | 6545 | 7633 |
| | 8035 | NA | 2.81E+11 | 9.52E+10 | 3.76E+11 | 75 | 25 | 8035 | 10755 |
| | 6088 | 88 | 1.98E+11 | 1.02E+10 | 2.08E+11 | 95 | 5 | 6088 | 6401 |
| | 5477 | 87 | 1.64E+11 | 2.02E+10 | 1.85E+11 | 89 | 11 | 5477 | 6151 |
| | 5998 | NA | 1.65E+11 | 8.61E+09 | 1.74E+11 | 95 | 5 | 5998 | 6311 |
| | 4036 | NA | 1.01E+11 | 7.83E+09 | 1.09E+11 | 93 | 7 | 4036 | 4349 |
| | 4195 | 83 | 9.44E+10 | 1.73E+09 | 9.61E+10 | 98 | 2 | 4195 | 4272 |
| | 8150 | | | | | | | | |
| | 1089 | | | | | | | | |
| | 2720 | | | | | | | | |
| | 313 | | | | | | | | |
| | 674 | | | | | | | | |
| | 313 | | | | | | | | |
| | 313 | | | | | | | | |
| | 77 | | | | | | | | |

TABLE 8
AEX-HPLC assay results of Ad5 and Ad11

| | | | AEX- | | AEX- | | | | |
|---|---|---|---|---|---|---|---|---|---|
| | | | HPLC | | HPLC | Vol- | | | |
| Virus | Infection | Sample | titer | | titer | ume | Total | Avg | Total |
| used | time (h) | Detail | vp/ml | DF | vp/ml | (ml) | vp | cells/ml | cells |
| Ad5 | 40 | SN 40h Ad5 | 0.00E+00 | 1.11 | 0.00E+00 | 40.00 | 0.00E+00 | 1.00E+06 | 4.00E+07 |
| | | D1D2 | | | | | | | |
| | 46 | SN 40h Ad5 | 0.00E+00 | 1.11 | 0.00E+00 | 37.50 | 0.00E+00 | 1.00E+06 | 3.75E+07 |
| | | D1D2 | | | | | | | |
| | 49 | SN 49h Ad5 | 0.00E+00 | 1.11 | 0.00E+00 | 35.00 | 0.00E+00 | 1.00E+06 | 3.50E+07 |
| | | D1D2 | | | | | | | |
| | 64 | SN 64h Ad5 | 0.00E+00 | 1.11 | 0.00E+00 | 32.50 | 0.00E+00 | 1.00E+06 | 3.25E+07 |
| | | D1D2 | | | | | | | |
| | 70 | SN 70h Ad5 | 0.00E+00 | 1.11 | 0.00E+00 | 30.00 | 0.00E+00 | 1.00E+06 | 3.00E+07 |
| | | D1D2 | | | | | | | |
| | 73 | SN 73h Ad5 | 0.00E+00 | 1.11 | 0.00E+00 | 27.50 | 0.00E+00 | 1.00E+06 | 2.75E+07 |
| | | D1D2 | | | | | | | |
| | 89 | SN 89h Ad5 | 0.00E+00 | 1.11 | 0.00E+00 | 25.00 | 0.00E+00 | 1.00E+06 | 2.50E+07 |
| | | D1D2 | | | | | | | |
| | 96 | SN 96h Ad5 | 0.00E+00 | 1.00 | 0.00E+00 | 22.50 | 0.00E+00 | 1.00E+06 | 2.25E+07 |
| | | D1D2 | | | | | | | |
| | 40 | CVL 40h Ad5 | 3.02E+10 | 0.44 | 1.34E+10 | 40.00 | 5.37E+11 | 1.00E+06 | 4.00E+07 |
| | | D1D2 | | | | | | | |
| | 46 | CVL 46h Ad5 | 3.86E+10 | 0.44 | 1.72E+10 | 37.50 | 6.44E+11 | 1.00E+06 | 3.75E+07 |
| | | D1D2 | | | | | | | |
| | 49 | CVL 49h Ad5 | 4.92E+10 | 0.44 | 2.18E+10 | 35.00 | 7.65E+11 | 1.00E+06 | 3.50E+07 |
| | | D1D2 | | | | | | | |
| | 64 | CVL 64h Ad5 | 3.84E+10 | 0.44 | 1.71E+10 | 32.50 | 5.54E+11 | 1.00E+06 | 3.25E+07 |
| | | D1D2 | | | | | | | |
| | 70 | CVL 70h Ad5 | 4.07E+10 | 0.44 | 1.81E+10 | 30.00 | 5.43E+11 | 1.00E+06 | 3.00E+07 |
| | | D1D2 | | | | | | | |
| | 73 | CVL 73h Ad5 | 3.45E+10 | 0.44 | 1.53E+10 | 27.50 | 4.21E+11 | 1.00E+06 | 2.75E+07 |
| | | D1D2 | | | | | | | |
| | 89 | CVL 89h Ad5 | 5.82E+10 | 0.44 | 2.59E+10 | 25.00 | 6.47E+11 | 1.00E+06 | 2.50E+07 |
| | | D1D2 | | | | | | | |
| | 96 | CVL 96h Ad5 | 2.35E+11 | 0.04 | 1.05E+10 | 22.50 | 2.35E+11 | 1.00E+06 | 2.25E+07 |
| | | D1D2 | | | | | | | |
| | | | | | Total | | | | Produced |
| | | | | | vp | Total | Total | | vp/cell |
| | | Cell | Total | Total | (SN | vp | vp | Produced | (SN |
| Virus | Produced | viability | vp | vp | + | (% in | (% in | vp/cell | + |
| used | vp/cell | % | (SN) | (CVL) | CVL) | SN) | CVL) | (in SN) | CVL) |
| Ad5 | 0 | 80 | 0.00E+00 | 5.37E+11 | 5.37E+11 | 0 | 100 | 0 | 13426 |
| | 0 | 89 | 0.00E+00 | 6.44E+11 | 6.44E+11 | 0 | 100 | 0 | 17165 |
| | 0 | NA | 0.00E+00 | 7.65E+11 | 7.65E+11 | 0 | 100 | 0 | 21846 |
| | 0 | 90 | 0.00E+00 | 5.54E+11 | 5.54E+11 | 0 | 100 | 0 | 17058 |
| | 0 | 89 | 0.00E+00 | 5.43E+11 | 5.43E+11 | 0 | 100 | 0 | 18086 |
| | 0 | NA | 0.00E+00 | 4.21E+11 | 4.21E+11 | 0 | 100 | 0 | 15322 |
| | 0 | NA | 0.00E+00 | 6.47E+11 | 6.47E+11 | 0 | 100 | 0 | 25885 |
| | 0 | 81 | 0.00E+00 | 2.35E+11 | 2.35E+11 | 0 | 100 | 0 | 10455 |
| | 13426 | | | | | | | | |
| | 17165 | | | | | | | | |
| | 21846 | | | | | | | | |
| | 17058 | | | | | | | | |
| | 18086 | | | | | | | | |
| | 15322 | | | | | | | | |
| | 25885 | | | | | | | | |
| | 10455 | | | | | | | | |
| | | | AEX- | | AEX- | | | | |
| | | | HPLC | | HPLC | Vol- | | | |
| Virus | Infection | Sample | titer | | titer | ume | Total | Avg | Total |
| used | time (h) | Detail | vp/ml | DF | vp/ml | (ml) | vp | cells/ml | cells |
| Ad11p | 40 | SN 40h Ad11 | 6.68E+09 | 1.11 | 7.41E+09 | 40.00 | 2.97E+11 | 1.00E+06 | 4.00E+07 |
| | | E1E2 | | | | | | | |
| | 46 | SN 46h Ad11 | 1.05E+10 | 1.11 | 1.16E+10 | 37.50 | 4.36E+11 | 1.00E+06 | 3.75E+07 |
| | | E1E2 | | | | | | | |
| | 49 | SN 490h Ad11 | 1.13E+10 | 1.11 | 1.25E+10 | 35.00 | 4.37E+11 | 1.00E+06 | 3.50E+07 |
| | | E1E2 | | | | | | | |
| | 64 | SN 64h Ad11 | 1.40E+10 | 1.11 | 1.55E+10 | 32.50 | 5.03E+11 | 1.00E+06 | 3.25E+07 |
| | | E1E2 | | | | | | | |
| | 70 | SN 70h Ad11 | 1.25E+10 | 1.11 | 1.39E+10 | 30.00 | 4.17E+11 | 1.00E+06 | 3.00E+07 |
| | | E1E2 | | | | | | | |
| | 73 | SN 73h Ad11 | 1.09E+10 | 1.11 | 1.21E+10 | 27.50 | 3.33E+11 | 1.00E+06 | 2.75E+07 |
| | | E1E2 | | | | | | | |
| | 89 | SN 89h Ad11 | 9.30E+09 | 1.11 | 1.03E+10 | 25.00 | 2.58E+11 | 1.00E+06 | 2.50E+07 |
| | | E1E2 | | | | | | | |
| | 96 | SN 96h Ad11 | 9.96E+09 | 1.00 | 9.96E+09 | 22.50 | 2.24E+11 | 1.00E+06 | 2.25E+07 |
| | | E1E2 | | | | | | | |
| | 40 | CVL 40h Ad11 | 8.37E+10 | 0.44 | 3.72E+10 | 40.00 | 1.49E+12 | 1.00E+06 | 4.00E+07 |
| | | E1E2 | | | | | | | |
| | 46 | CVL 46h Ad11 | 6.23E+10 | 0.44 | 2.77E+10 | 37.50 | 1.04E+12 | 1.00E+06 | 3.75E+07 |
| | | E1E2 | | | | | | | |
| | 49 | CVL 49h Ad11 | 8.80E+10 | 0.44 | 3.91E+10 | 35.00 | 1.37E+12 | 1.00E+06 | 3.50E+07 |
| | | E1E2 | | | | | | | |
| | 64 | CVL 64h Ad11 | 1.05E+10 | 0.44 | 4.68E+09 | 32.50 | 1.52E+11 | 1.00E+06 | 3.25E+07 |
| | | E1E2 | | | | | | | |
| | 70 | CVL 70h Ad11 | 8.72E+09 | 0.44 | 3.88E+09 | 30.00 | 1.16E+11 | 1.00E+06 | 3.00E+07 |
| | | E1E2 | | | | | | | |
| | 73 | CVL 73h Ad11 | 6.33E+09 | 0.44 | 2.81E+09 | 27.50 | 7.74E+10 | 1.00E+06 | 2.75E+07 |
| | | E1E2 | | | | | | | |
| | 89 | CVL 89h Ad11 | 2.21E+09 | 0.44 | 9.81E+08 | 25.00 | 2.45E+10 | 1.00E+06 | 2.50E+07 |
| | | E1E2 | | | | | | | |
| | 96 | CVL 96h Ad11 | 1.10E+10 | 0.04 | 4.89E+08 | 22.50 | 1.10E+10 | 1.00E+06 | 2.25E+07 |
| | | E1E2 | | | | | | | |
| | | | | | Total | | | | Produced |
| | | | | | vp | Total | Total | | vp/cell |
| | | Cell | Total | Total | (SN | vp | vp | Produced | (SN |
| Virus | Produced | viability | vp | vp | + | (% in | (% in | vp/cell | + |
| used | vp/cell | % | (SN) | (CVL) | CVL) | SN) | CVL) | (in SN) | CVL) |
| Ad11p | 7415 | 73 | 2.97E+11 | 1.49E+12 | 1.78E+12 | 17 | 83 | 7415 | 44601 |
| | 11639 | 91 | 4.36E+11 | 1.04E+12 | 1.48E+12 | 30 | 70 | 11639 | 39340 |
| | 12498 | NA | 4.37E+11 | 1.37E+12 | 1.81E+12 | 24 | 76 | 12498 | 51629 |
| | 15492 | 89 | 5.03E+11 | 1.52E+11 | 6.56E+11 | 77 | 23 | 15492 | 20170 |
| | 13891 | 89 | 4.17E+11 | 1.16E+11 | 5.33E+11 | 78 | 22 | 13891 | 17768 |
| | 12098 | NA | 3.33E+11 | 7.74E+10 | 4.10E+11 | 81 | 19 | 12098 | 14913 |
| | 10320 | NA | 2.58E+11 | 2.45E+10 | 2.83E+11 | 91 | 9 | 10320 | 11301 |
| | 9965 | 85 | 2.24E+11 | 1.10E+10 | 2.35E+11 | 95 | 5 | 9965 | 10453 |
| | 37186 | | | | | | | | |
| | 27702 | | | | | | | | |
| | 39130 | | | | | | | | |
| | 4678 | | | | | | | | |
| | 3877 | | | | | | | | |
| | 2815 | | | | | | | | |
| | 981 | | | | | | | | |
| | 489 | | | | | | | | |

1. A process for the manufacture of a chimeric oncolytic adenovirus having a genome comprising an E2B region, wherein said E2B region comprises a nucleic acid sequence derived from a first adenoviral serotype and a nucleic acid sequence derived from a second distinct adenoviral serotype; wherein said first and second serotypes are each selected from the adenoviral subgroups B, C, D, E, or F, wherein the virus has a hexon and fibre from a group B adenovirus, said virus does not express a death protein from an E3 region in the virus and said virus is replication competent; wherein the process comprises the steps:
a. culturing mammalian cells infected with the adenovirus in the presence of media suitable for supporting the cells such that the virus replicates, wherein the cells are capable of supporting viral replication, and
b. at the end of the culturing period isolating from the media the virus from step a) by filtering wherein the isolation of virus is not subsequent to a cell lysis step, wherein the culturing period is in the range of 30 to 100 hours.

2. A process according to claim 1, wherein the virus has a hexon and fibre from Ad11.

3. A process according to claim 2, wherein the virus is ColoAd1.

4. A process according to 1 to claim 3, wherein the culturing period is in the range 35 to 80 hours.

5. A process according to claim 1, wherein the culturing comprises a perfusion culture step, fed batch, or batch.

6. A process according to claim 1, wherein the cells are grown in suspension culture.

7. A process according to claim 1, wherein the mammalian cells are selected from the group consisting of HEK, CHO, COS-7 HeLa, Viro, A549 , PerC6 and GMK.

8. A process according to claim 1, wherein the culture is a scale of 5 L or more.

9. A process according to claim 1, wherein virus during culture is at a concentration in the range 40 to 150 ppc.

10. A process according to claim 1, wherein the cells are infected with a starting concentration of virus of 1-9×10⁴ vp/ml or greater.

11. A process according to claim 1, which provides a fraction of oncolytic virus, wherein the process comprises a further step such that a second fraction or fractions of the oncolytic virus made by the same or a different process is/are combined with the first fraction.

12. A process according to claim 1, wherein the process is a GMP manufacturing process.

13. A process according to claim 1, wherein the filter is a tangential filter.

14. A process according to claim 1, wherein the process further comprises a purification stepₛ selected from the group consisting of a CsCl gradient and a chromatography step, and a combination thereof.

15. A process according to claim 1, wherein 40 to 93% of the total virus is recoverable from the media.

16. A process according to claim 1, which further comprises formulating the virus in a buffer suitable for storage.

17. (canceled)

18. A process according to claim 3, wherein the virus further comprises a transgene.

19. A process according to claim 8, wherein the cells are infected with virus at a starting concentration in the range 20 to 150 ppc.

20. A process for the manufacture of an Ad11 adenovirus, comprising steps of:
a. culturing mammalian cells infected with the adenovirus in the presence of media suitable for supporting the cells such that the virus replicates, wherein the cells are capable of supporting viral replication, and
b. at the end of the culturing period isolating form the media the adenovirus from step a) by filtering, wherein the isolation of virus is not subsequent to a cell lysis step, wherein the Ad11 virus does not express a death protein from an E3 region in the virus.


SEQUENCE LISTING (2 sequences)

SEQ ID NO: 1 - DNA, 32326 bp, ColoAd1 adenovirus
tctatctatataatataccttatagatggaatggtgccaatatgtaaatgaggtgattttaaaaagtgtggatcgtgtggtgattggctgtggggttaacggctaaaaggggcggtgcgaccgtgggaaaatgacgttttgtgggggtggagtttttttgcaagttgtcgcgggaaatgtgacgcataaaaaggcttttttctcacggaactacttagttttcccacggtatttaacaggaaatgaggtagttttgaccggatgcaagtgaaaattgttgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatttatacctcagggtttgtgtcaagaggccactcttgagtgccagcgagaagagttttctcctctgcgccggcagtttaataataaaaaaatgagagatttgcgatttctgcctcaggaaataatctctgctgagactggaaatgaaatattggagcttgtggtgcacgccctgatgggagacgatccggagccacctgtgcagctttttgagcctcctacgcttcaggaactgtatgatttagaggtagagggatcggaggattctaatgaggaagctgtaaatggcttttttaccgattctatgcttttagctgctaatgaagggttagaattagatccgcctttggacacttttgatactccaggggtaattgtggaaagcggtacaggtgtaagaaaattacctgatttgagttccgtggactgtgatttgcactgctatgaagacgggtttcctccgagtgatgaggaggaccatgaaaaggagcagtccatgcagactgcagcgggtgagggagtgaaggctgccaatgttggttttcagttggattgcccggagcttcctggacatggctgtaagtcttgtgaatttcacaggaaaaatactggagtaaaggaactgttatgttcgctttgttatatgagaacgcactgccactttatttacagtaagtgtgtttaagttaaaatttaaaggaatatgctgtttttcacatgtatattgagtgtgagttttgtgcttcttattataggtcctgtgtctgatgctgatgaatcaccatctcctgattctactacctcacctcctgagattcaagcacctgttcctgtggacgtgcgcaagcccattcctgtgaagcttaagcctgggaaacgtccagcagtggaaaaacttgaggacttgttacagggtggggacggacctttggacttgagtacacggaaacgtccaagacaataagtgttccatatccgtgtttacttaaggtgacgtcaatatttgtgtgacagtgcaatgtaataaaaatatgttaactgttcactggtttttattgctttttgggcggggactcaggtatataagtagaagcagacctgtgtggttagctcataggagctggctttcatccatggaggtttgggccattttggaagaccttaggaagactaggcaactgttagagaacgcttcggacggagtctccggtttttggagattctggttcgctagtgaattagctagggtagtttttaggataaaacaggactataaacaagaatttgaaaagttgttggtagattgcccaggactttttgaagctcttaatttgggccatcaggttcactttaaagaaaaagttttatcagttttagacttttcaaccccaggtagaactgctgctgctgtggcttttcttacttttatattagataaatggatcccgcagactcatttcagcaggggatacgttttggatttcatagccacagcattgtggagaacatggaaggttcgcaagatgaggacaatcttaggttactggccagtgcagcctttgggtgtagcgggaatcctgaggcatccaccggtcatgccagcggttctggaggaggaacagcaagaggacaacccgagagccggcctggaccctccagtggaggaggcggagtagctgacttgtctcctgaactgcaacgggtgcttactggatctacgtccactggacgggataggggcgttaagagggagagggcatctagtggtactgatgctagatctgagttggctttaagtttaatgagtcgcagacgtcctgaaaccatttggtggcatgaggttcagaaagagggaagggatgaagtttctgtattgcaggagaaatattcactggaacaggtgaaaacatgttggttggagcctgaggatgattgggaggtggccattaaaaattatgccaagatagctttgaggcctgataaacagtataagattactagacggattaatatccggaatgcttgttacatatctggaaatggggctgaggtggtaatagatactcaagacaaggcagttattagatgctgcatgatggatatgtggcctggggtagtcggtatggaagcagtaacttttgtaaatgttaagtttaggggagatggttataatggaatagtgtttatggccaataccaaacttatattgcatggttgtagcttttttggtttcaacaatacctgtgtagatgcctggggacaggttagtgtacggggatgtagtttctatgcgtgttggattgccacagctggcagaaccaagagtcaattgtctctgaagaaatgcatatttcaaagatgtaacctgggcattctgaatgaaggcgaagcaagggtccgccactgcgcttctacagatactggatgttttattttgattaagggaaatgccagcgtaaagcataacatgatttgcggtgcttccgatgagaggccttatcaaatgctcacttgtgctggtgggcattgtaatatgctggctactgtgcatattgtttcccatcaacgcaaaaaatggcctgtttttgatcacaatgtgatgacgaagtgtaccatgcatgcaggtgggcgtagaggaatgtttatgccttaccagtgtaacatgaatcatgtgaaagtgttgttggaaccagatgccttttccagaatgagcctaacaggaatttttgacatgaacatgcaaatctggaagatcctgaggtatgatgatacgagatcgagggtacgcgcatgcgaatgcggaggcaagcatgccaggttccagccggtgtgtgtagatgtgactgaagatctcagaccggatcatttggttattgcccgcactggagcagagttcggatccagtggagaagaaactgactaaggtgagtattgggaaaactttggggtgggattttcagatggacagattgagtaaaaatttgttttttctgtcttgcagctgtcatgagtggaaacgcttcttttaaggggggagtcttcagcccttatctgacagggcgtctcccatcctgggcaggagttcgtcagaatgttatgggatctactgtggatggaagacccgtccaacccgccaattcttcaacgctgacctatgctactttaagttcttcacctttggacgcagctgcagctgccgccgccgcttctgttgccgctaacactgtgcttggaatgggttactatggaagcatcatggctaattccacttcctctaataacccttctaccctgactcaggacaagttacttgtccttttggcccagctggaggctttgacccaacgtctgggtgaactttctcagcaggtggtcgagttgcgagtacaaactgagtctgctgtcggcacggcaaagtctaaataaaaaaatcccagaatcaatgaataaataaacaagcttgttgttgatttaaaatcaagtgtttttatttcatttttcgcgcacggtatgccctagaccaccgatctctatcattgagaactcggtggattttttccaggatcctatagaggtgggattgaatgtttagatacatgggcattaggccgtctttggggtggagatagctccattgaagggattcatgctccggggtagtgttgtaaatcacccagtcataacaaggtcgcagtgcatggtgttgcacaatatcttttagaagtaggctgattgccacagataagcccttggtgtaggtgtttacaaaccggttgagctgggatgggtgcattcggggtgaaattatgtgcattttggattggatttttaagttggcaatattgccgccaagatcccgtcttgggttcatgttatgaaggaccaccaagacggtgtatccggtacatttaggaaatttatcgtgcagcttggatggaaaagcgtggaaaaatttggagacacccttgtgtcctccaagattttccatgcactcatccatgataatagcaatggggccgtgggcagcggcgcgggcaaacacgttccgtgggtctgacacatcatagttatgttcctgagttaaatcatcataagccattttaatgaatttggggcggagagtaccagattggggtatgaatgttccttcgggccccggagcatagttcccctcacagatttgcatttcccaagctttcagttccgagggtggaatcatgtccacctggggggctatgaaaaacaccgtttctggggcgggggtgattaattgtgatgatagcaaatttctgagcaattgagatttgccacatccggtggggccataaatgattccgattacgggttgcaggtggtagtttagggaacggcaactgccgtcttctcgaagcaagggggccacctcgttcatcatttcccttacatgcatattttcccgcaccaaatccattaggaggcgctctcctcctagtgatagaagttcttgtagtgaggaaaagtttttcagcggtttcagaccgtcagccatgggcattttggagagagtttgctgcaaaagttctagtctgttccacagttcagtgatgtgttctatggcatctcgatccagcagacctcctcgtttcgcgggtttggacggctcctggaatagggtatgagacgatgggcgtccagcgctgccagggttcggtccttccagggtctcagtgttcgagtcagggttgtttccgtcacagtgaaggggtgtgcgcctgcttgggcgcttgccagggtgcgcttcagactcatcctgctggtcgaaaacttctgtcgcttggcgccctgtatgtcggccaagtagcagtttaccatgagttcgtagttgagcgcctcggctgcgtggcctttggcgcggagcttacctttggaagttttcttgcataccgggcagtataggcatttcagcgcatacaacttgggcgcaaggaaaacggattctggggagtatgcatctgcgccgcaggaggcgcaaacagtttcacattccaccagccaggttaaatccggttcattggggtcaaaaacaagttttccgccatattttttgatgcgtttcttacctttggtctccatgagttcgtgtcctcgttgagtgacaaacaggctgtccgtgtccccgtagactgattttacaggcctcttctccagtggagtgcctcggtcttcttcgtacaggaactctgaccactctgatacaaaggcgcgcgtccaggccagcacaaaggaggctatgtgggaggggtagcgatcgttgtcaaccagggggtccaccttttccaaagtatgcaaacacatgtcaccctcttcaacatccaggaatgtgattggcttgtaggtgtatttcacgtgacctggggtccccgctgggggggtataaaagggggcggttctttgctcttcctcactgtcttccggatcgctgtccaggaacgtcagctgttggggtaggtattccctctcgaaggcgggcatgacctctgcactcaggttgtcagtttctaagaacgaggaggatttgatattgacagtgccggttgagatgcctttcatgaggttttcgtccatctggtcagaaaacacaatttttttattgtcaagtttggtggcaaatgatccatacagggcgttggataaaagtttggcaatggatcgcatggtttggttcttttccttgtccgcgcgctctttggcggcgatgttgagttggacatactcgcgtgccaggcacttccattcggggaagatagttgttaattcatctggcacgattctcacttgccaccctcgattatgcaaggtaattaaatccacactggtggccacctcgcctcgaaggggttcattggtccaacagagcctacctcctttcctagaacagaaagggggaagtgggtctagcataagttcatcgggagggtctgcatccatggtaaagattcccggaagtaaatccttatcaaaatagctgatgggagtggggtcatctaaggccatttgccattctcgagctgccagtgcgcgctcatatgggttaaggggactgccccatggcatgggatgggtgagtgcagaggcatacatgccacagatgtcatagacgtagatgggatcctcaaagatgcctatgtaggttggatagcatcgcccccctctgatacttgctcgcacatagtcatatagttcatgtgatggcgctagcagccccggacccaagttggtgcgattgggtttttctgttctgtagacgatctggcgaaagatggcgtgagaattggaagagatggtgggtctttgaaaaatgttgaaatgggcatgaggtagacctacagagtctctgacaaagtgggcataagattcttgaagcttggttaccagttcggcggtgacaagtacgtctagggcgcagtagtcaagtgtttcttgaatgatgtcataacctggttggtttttcttttcccacagttcgcggttgagaaggtattcttcgcgatccttccagtactcttctagcggaaacccgtctttgtctgcacggtaagatcctagcatgtagaactgattaactgccttgtaagggcagcagcccttctctacgggtagagagtatgcttgagcagcttttcgtagcgaagcgtgagtaagggcaaaggtgtctctgaccatgactttgaggaattggtatttgaagtcgatgtcgtcacaggctccctgttcccagagttggaagtctacccgtttcttgtaggcggggttgggcaaagcgaaagtaacatcattgaagagaatcttgccggccctgggcatgaaattgcgagtgatgcgaaaaggctgtggtacttccgctcggttattgataacctgggcagctaggacgatctcgtcgaaaccgttgatgttgtgtcctacgatgtataattctatgaaacgcggcgtgcctctgacgtgaggtagcttactgagctcatcaaaggttaggtctgtggggtcagataaggcgtagtgttcgagagcccattcgtgcaggtgaggattcgctttaaggaaggaggaccagaggtccactgccagtgctgtttgtaactggtcccggtactgacgaaaatgccgtccgactgccattttttctggggtgacgcaatagaaggtttgggggtcctgccgccagcgatcccacttgagttttatggcgaggtcataggcgatgttgacgagccgctggtctccagagagtttcatgaccagcatgaaggggattagctgcttgccaaaggaccccatccaggtgtaggtttccacatcgtaggtgagaaagagcctttctgtgcgaggatgagagccaatcgggaagaactggatctcctgccaccagttggaggaatggctgttgatgtgatggaagtagaactccctgcgacgcgccgagcattcatgcttgtgcttgtacagacggccgcagtagtcgcagcgttgcacgggttgtatctcgtgaatgagttgtacctggcttcccttgacgagaaatttcagtgggaagccgaggcctggcgattgtatctcgtgctttactatgttgtctgcatcggcctgttcatcttctgtctcgatggtggtcatgctgacgagccctcgcgggaggcaagtccagacctcggcgcggcaggggcggagctcgaggacgagagcgcgcaggctggagctgtccagggtcctgagacgctgcggactcaggttagtaggcagtgtcaggagattaacttgcatgatcttttggagggcgtgcgggaggttcagatagtacttgatctcaacgggtccgttggtggagatgtcgatggcttgcagggttccgtgtcccttgggcgctaccaccgtgcccttgtttttcattttggacggcggtggctctgttgcttcttgcatgtttagaagcggtgtcgagggcgcgcaccgggcggcaggggcggctcgggacccggcggcatggctggcagtggtacgtcggcgccgcgcgcgggtaggttctggtactgcgccctgagaagactcgcatgcgcgacgacgcggcggttgacatcctggatctgacgcctctgggtgaaagctaccggccccgtgagcttgaacctgaaagagagttcaacagaatcaatctcggtatcgttgacggcggcttgcctaaggatttcttgcacgtcaccagagttgtcctggtaggcgatctccgccatgaactgctcgatctcttcctcttgaagatctccgcggcccgctctctcgacggtggccgcgaggtcgttggagatgcgcccaatgagttgagagaatgcattcatgcccgcctcgttccagacgcggctgtagaccacggcccccacgggatctctcgcgcgcatgaccacctgggcgaggttgagctccacgtggcgggtgaagaccgcatagttgcataggcgctggaaaaggtagttgagtgtggtggcgatgtgctcggtgacgaagaaatacatgatccatcgtctcagcggcatctcgctgacatcgcccagagcttccaagcgctccatggcctcgtagaagtccacggcaaaattaaaaaactgggagtttcgcgcggacacggtcaactcctcttccagaagacggataagttcggcgatggtggtgcgcacctcgcgctcgaaagcccctgggatttcttcctcaatctcttcttcttccactaacatctcttcctcttcaggtggggctgcaggaggagggggaacgcggcgacgccggcggcgcacgggcagacggtcgatgaatctttcaatgacctctccgcggcggcggcgcatggtttcagtgacggcgcggccgttctcgcgcggtcgcagagtaaaaacaccgccgcgcatctccttaaagtggtgactgggaggttctccgtttgggagggagagggcgctgattatacattttattaattggcccgtagggactgcacgcagagatctgatcgtgtcaagatccacgggatctgaaaacctttcgacgaaagcgtctaaccagtcacagtcacaaggtaggctgagtacggcttcttgtgggcgggggtggttatgtgttcggtctgggtcttctgtttcttcttcatctcgggaaggtgagacgatgctgctggtgatgaaattaaagtaggcagttctaagacggcggatggtggcgaggagcaccaggtctttgggtccggcttgctggatacgcaggcgattggccattccccaagcattatcctgacatctagcaagatctttgtagtagtcttgcatgagccgttctacgggcacttcttcctcacccgttctgccatgcatacgtgtgagtccaaatccgcgcattggttgtaccagtgccaagtcagctacgactctttcggcgaggatggcttgctgtacttgggtaagggtggcttgaaagtcatcaaaatccacaaagcggtggtaagctcctgtattaatggtgtaagcacagttggccatgactgaccagttaactgtctggtgaccagggcgcacgagctcggtgtatttaaggcgcgaataggcgcgggtgtcaaagatgtaatcgttgcaggtgcgcaccagatactggtaccctataagaaaatgcggcggtggttggcggtagagaggccatcgttctgtagctggagcgccaggggcgaggtcttccaacataaggcggtgatagccgtagatgtacctggacatccaggtgattcctgcggcggtagtagaagcccgaggaaactcgcgtacgcggttccaaatgttgcgtagcggcatgaagtagttcattgtaggcacggtttgaccagtgaggcgcgcgcagtcattgatgctctatagacacggagaaaatgaaagcgttcagcgactcgactccgtagcctggaggaacgtgaacgggttgggtcgcggtgtaccccggttcgagacttgtactcgagccggccggagccgcggctaacgtggtattggcactcccgtctcgacccagcctacaaaaatccaggatacggaatcgagtcgttttgctggtttccgaatggcagggaagtgagtcctattttttttttttgccgctcagatgcatcccgtgctgcgacagatgcgcccccaacaacagcccccctcgcagcagcagcagcagcaatcacaaaaggctgtccctgcaactactgcaactgccgccgtgagcggtgcgggacagcccgcctatgatctggacttggaagagggcgaaggactggcacgtctaggtgcgccttcacccgagcggcatccgcgagttcaactgaaaaaagattctcgcgaggcgtatgtgccccaacagaacctatttagagacagaagcggcgaggagccggaggagatgcgagcttcccgctttaacgcgggtcgtgagctgcgtcacggtttggaccgaagacgagtgttgcgggacgaggatttcgaagttgatgaaatgacagggatcagtcctgccagggcacacgtggctgcagccaaccttgtatcggcttacgagcagacagtaaaggaagagcgtaacttccaaaagtcttttaataatcatgtgcgaaccctgattgcccgcgaagaagttacccttggtttgatgcatttgtgggatttgatggaagctatcattcagaaccctactagcaaacctctgaccgcccagctgtttctggtggtgcaacacagcagagacaatgaggctttcagagaggcgctgctgaacatcaccgaacccgaggggagatggttgtatgatcttatcaacattctacagagtatcatagtgcaggagcggagcctgggcctggccgagaaggtggctgccatcaattactcggttttgagcttgggaaaatattacgctcgcaaaatctacaagactccatacgttcccatagacaaggaggtgaagatagatgggttctacatgcgcatgacgctcaaggtcttgaccctgagcgatgatcttggggtgtatcgcaatgacagaatgcatcgcgcggttagcgccagcaggaggcgcgagttaagcgacagggaactgatgcacagtttgcaaagagctctgactggagctggaaccgagggtgagaattacttcgacatgggagctgacttgcagtggcagcctagtcgcagggctctgagcgccgcgacggcaggatgtgagcttccttacatagaagaggcggatgaaggcgaggaggaagagggcgagtacttggaagactgatggcacaacccgtgttttttgctagatggaacagcaagcaccggatcccgcaatgcgggcggcgctgcagagccagccgtccggcattaactcctcggacgattggacccaggccatgcaacgtatcatggcgttgacgactcgcaaccccgaagcctttagacagcaaccccaggccaaccgtctatcggccatcatggaagctgtagtgccttcccgctctaatcccactcatgagaaggtcctggccatcgtgaacgcgttggtggagaacaaagctattcgtccagatgaggccggactggtatacaacgctctcttagaacgcgtggctcgctacaacagtagcaatgtgcaaaccaatttggaccgtatgataacagatgtacgcgaagccgtgtctcagcgcgaaaggttccagcgtgatgccaacctgggttcgctggtggcgttaaatgctttcttgagtactcagcctgctaatgtgccgcgtggtcaacaggattatactaactttttaagtgctttgagactgatggtatcagaagtacctcagagcgaagtgtatcagtccggtcctgattacttctttcagactagcagacagggcttgcagacggtaaatctgagccaagcttttaaaaaccttaaaggtttgtggggagtgcatgccccggtaggagaaagagcaaccgtgtctagcttgttaactccgaactcccgcctattattactgttggtagctcctttcaccgacagcggtagcatcgaccgtaattcctatttgggttacctactaaacctgtatcgcgaagccatagggcaaagtcaggtggacgagcagacctatcaagaaattacccaagtcagtcgcgctttgggacaggaagacactggcagtttggaagccactctgaacttcttgcttaccaatcggtctcaaaagatccctcctcaatatgctcttactgcggaggaggagaggatccttagatatgtgcagcagagcgtgggattgtttctgatgcaagagggggcaactccgactgcagcactggacatgacagcgcgaaatatggagcccagcatgtatgccagtaaccgacctttcattaacaaactgctggactacttgcacagagctgccgctatgaactctgattatttcaccaatgccatcttaaacccgcactggctgcccccacctggtttctacacgggcgaatatgacatgcccgaccctaatgacggatttctgtgggacgacgtggacagcgatgttttttcacctctttctgatcatcgcacgtggaaaaaggaaggcggcgatagaatgcattcttctgcatcgctgtccggggtcatgggtgctaccgcggctgagcccgagtctgcaagtccttttcctagtctacccttttctctacacagtgtacgtagcagcgaagtgggtagaataagtcgcccgagtttaatgggcgaagaggagtatctaaacgattccttgctcagaccggcaagagaaaaaaatttcccaaacaatggaatagaaagtttggtggataaaatgagtagatggaagacttatgctcaggatcacagagacgagcctgggatcatggggattacaagtagagcgagccgtagacgccagcgccatgacagacagaggggtcttgtgtgggacgatgaggattcggccgatgatagcagcgtgctggacttgggtgggagaggaaggggcaacccgtttgctcatttgcgccctcgcttgggtggtatgttgtaaaaaaaaataaaaaaaaaactcaccaaggccatggcgacgagcgtacgttcgttcttctttattatctgtgtctagtataatgaggcgagtcgtgctaggcggagcggtggtgtatccggagggtcctcctccttcgtacgagagcgtgatgcagcagcagcaggcgacggcggtgatgcaatccccactggaggctccctttgtgcctccgcgatacctggcacctacggagggcagaaacagcattcgttattcggaactggcacctcagtacgataccaccaggttgtatctggtggacaacaagtcggcggacattgcttctctgaactatcagaatgaccacagcaacttcttgaccacggtggtgcaaaacaatgactttacccctacggaagccagcacccagaccattaactttgatgaacgatcgcggtggggcggtcagctaaagaccatcatgcatactaacatgccaaacgtgaacgagtatatgtttagtaacaagttcaaagcgcgtgtgatggtgtccagaaaacctcccgacggtgctgcagttggggatacttatgatcacaagcaggatattttgaaatatgagtggttcgagtttactttgccagaaggcaacttttcagttactatgactattgatttgatgaacaatgccatcatagataattacttgaaagtgggtagacagaatggagtgcttgaaagtgacattggtgttaagttcgacaccaggaacttcaagctgggatgggatcccgaaaccaagttgatcatgcctggagtgtatacgtatgaagccttccatcctgacattgtcttactgcctggctgcggagtggattttaccgagagtcgtttgagcaaccttcttggtatcagaaaaaaacagccatttcaagagggttttaagattttgtatgaagatttagaaggtggtaatattccggccctcttggatgtagatgcctatgagaacagtaagaaagaacaaaaagccaaaatagaagctgctacagctgctgcagaagctaaggcaaacatagttgccagcgactctacaagggttgctaacgctggagaggtcagaggagacaattttgcgccaacacctgttccgactgcagaatcattattggccgatgtgtctgaaggaacggacgtgaaactcactattcaacctgtagaaaaagatagtaagaatagaagctataatgtgttggaagacaaaatcaacacagcctatcgcagttggtatctttcgtacaattatggcgatcccgaaaaaggagtgcgttcctggacattgctcaccacctcagatgtcacctgcggagcagagcaggtctactggtcgcttccagacatgatgaaggatcctgtcactttccgctccactagacaagtcagtaactaccctgtggtgggtgcagagcttatgcccgtcttctcaaagagcttctacaacgaacaagctgtgtactcccagcagctccgccagtccacctcgcttacgcacgtcttcaaccgctttcctgagaaccagattttaatccgtccgccggcgcccaccattaccaccgtcagtgaaaacgttcctgctctcacagatcacgggaccctgccgttgcgcagcagtatccggggagtccaacgtgtgaccgttactgacgccagacgccgcacctgtccctacgtgtacaaggcactgggcatagtcgcaccgcgcgtcctttcaagccgcactttctaaaaaaaaaaaaaatgtccattcttatctcgcccagtaataacaccggttggggtctgcgcgctccaagcaagatgtacggaggcgcacgcaaacgttctacccaacatcctgtccgtgttcgcggacattttcgcgctccatggggcgccctcaagggccgcactcgcgttcgaaccaccgtcgatgatgtaatcgatcaggtggttgccgacgcccgtaattatactcctactgcgcctacatctactgtggatgcagttattgacagtgtagtggctgacgctcgcaactatgctcgacgtaagagccggcgaaggcgcattgccagacgccaccgagctaccactgccatgcgagccgcaagagctctgctacgaagagctagacgcgtggggcgaagagccatgcttagggcggccagacgtgcagcttcgggcgccagcgccggcaggtcccgcaggcaagcagccgctgtcgcagcggcgactattgccgacatggcccaatcgcgaagaggcaatgtatactgggtgcgtgacgctgccaccggtcaacgtgtacccgtgcgcacccgtccccctcgcacttagaagatactgagcagtctccgatgttgtgtcccagcggcgaggatgtccaagcgcaaatacaaggaagaaatgctgcaggttatcgcacctgaagtctacggccaaccgttgaaggatgaaaaaaaaccccgcaaaatcaagcgggttaaaaaggacaaaaaagaagaggaagatggcgatgatgggctggcggagtttgtgcgcgagtttgccccacggcgacgcgtgcaatggcgtgggcgcaaagttcgacatgtgttgagacctggaacttcggtggtctttacacccggcgagcgttcaagcgctacttttaagcgttcctatgatgaggtgtacggggatgatgatattcttgagcaggcggctgaccgattaggcgagtttgcttatggcaagcgtagtagaataacttccaaggatgagacagtgtcgatacccttggatcatggaaatcccacccctagtcttaaaccggtcactttgcagcaagtgttacccgtaactccgcgaacaggtgttaaacgcgaaggtgaagatttgtatcccactatgcaactgatggtacccaaacgccagaagttggaggacgttttggagaaagtaaaagtggatccagatattcaacctgaggttaaagtgagacccattaagcaggtagcgcctggtctgggggtacaaactgtagacattaagattcccactgaaagtatggaagtgcaaactgaacccgcaaagcctactgccacctccactgaagtgcaaacggatccatggatgcccatgcctattacaactgacgccgccggtcccactcgaagatcccgacgaaagtacggtccagcaagtctgttgatgcccaattatgttgtacacccatctattattcctactcctggttaccgaggcactcgctactatcgcagccgaaacagtacctcccgccgtcgccgcaagacacctgcaaatcgcagtcgtcgccgtagacgcacaagcaaaccgactcccggcgccctggtgcggcaagtgtaccgcaatggtagtgcggaacctttgacactgccgcgtgcgcgttaccatccgagtatcatcacttaatcaatgttgccgctgcctccttgcagatatggccctcacttgtcgccttcgcgttcccatcactggttaccgaggaagaaactcgcgccgtagaagagggatgttgggacgcggaatgcgacgctacaggcgacggcgtgctatccgcaagcaattgcggggtggttttttaccagccttaattccaattatcgctgctgcaattggcgcgataccaggcatagcttccgtggcggttcaggcctcgcaacgacattgacattggaaaaaaacgtataaataaaaaaaaaaaaatacaatggactctgacactcctggtcctgtgactatgttttcttagagatggaagacatcaatttttcatccttggctccgcgacacggcacgaagccgtacatgggcacctggagcgacatcggcacgagccaactgaacgggggcgccttcaattggagcagtatctggagcgggcttaaaaattttggctcaaccataaaaacatacgggaacaaagcttggaacagcagtacaggacaggcgcttagaaataaacttaaagaccagaacttccaacaaaaagtagtcgatgggatagcttccggcatcaatggagtggtagatttggctaaccaggctgtgcagaaaaagataaacagtcgtttggacccgccgccagcaaccccaggtgaaatgcaagtggaggaagaaattcctccgccagaaaaacgaggcgacaagcgtccgcgtcccgatttggaagagacgctggtgacgcgcgtagatgaaccgccttcttatgaggaagcaacgaagcttggaatgcccaccactagaccgatagccccaatggccaccggggtgatgaaaccttctcagttgcatcgacccgtcaccttggatttgccccctccccctgctgctactgctgtacccgcttctaagcctgtcgctgccccgaaaccagtcgccgtagccaggtcacgtcccgggggcgctcctcgtccaaatgcgcactggcaaaatactctgaacagcatcgtgggtctaggcgtgcaaagtgtaaaacgccgtcgctgcttttaattaaatatggagtagcgcttaacttgcctatctgtgtatatgtgtcattacacgccgtcacagcagcagaggaaaaaaggaagaggtcgtgcgtcgacgctgagttactttcaagatggccaccccatcgatgctgccccaatgggcatacatgcacatcgccggacaggatgcttcggagtacctgagtccgggtctggtgcagttcgcccgcgccacagacacctacttcaatctgggaaataagtttagaaatcccaccgtagcgccgacccacgatgtgaccaccgaccgtagccagcggctcatgttgcgcttcgtgcccgttgaccgggaggacaatacatactcttacaaagtgcggtacaccctggccgtgggcgacaacagagtgctggatatggccagcacgttctttgacattaggggtgtgttggacagaggtcccagtttcaaaccctattctggtacggcttacaactccctggctcctaaaggcgctccaaatacatctcagtggattgcagaaggtgtaaaaaatacaactggtgaggaacacgtaacagaagaggaaaccaatactactacttacacttttggcaatgctcctgtaaaagctgaagctgaaattacaaaagaaggactcccagtaggtttggaagtttcagatgaagaaagtaaaccgatttatgctgataaaacatatcagccagaacctcagctgggagatgaaacttggactgaccttgatggaaaaaccgaaaagtatggaggcagggctctcaaacccgatactaagatgaaaccatgctacgggtcctttgccaaacctactaatgtgaaaggcggtcaggcaaaacaaaaaacaacggagcagccaaatcagaaagtcgaatatgatatcgacatggagttttttgatgcggcatcgcagaaaacaaacttaagtcctaaaattgtcatgtatgcagaaaatgtaaatttggaaactccagacactcatgtagtgtacaaacctggaacagaagacacaagttccgaagctaatttgggacaacaatctatgcccaacagacccaactacattggcttcagagataactttattggacttatgtactataacagtactggtaacatgggggtgctggctggtcaagcgtctcagttaaatgcagtggttgacttgcaggacagaaacacagaactttcttaccaactcttgcttgactctctgggcgacagaaccagatactttagcatgtggaatcaggctgtggacagttatgatcctgatgtacgtgttattgaaaatcatggtgtggaagatgaacttcccaactactgttttccactggacggcataggtgttccaacaaccagttacaaatcaatagttccaaatggagacaatgcgcctaattggaaggaacctgaagtaaatggaacaagtgagatcggacagggtaatttgtttgccatggaaattaaccttcaagccaatctatggcgaagtttcctttattccaatgtggctctatatctcccagactcgtacaaatacaccccgtccaatgtcactcttccagaaaacaaaaacacctacgactacatgaacgggcgggtggtgccgccatctctagtagacacctatgtgaacattggtgccaggtggtctctggatgccatggacaatgtcaacccattcaaccaccaccgtaacgctggcttgcgttaccgatccatgcttctgggtaacggacgttatgtgcctttccacatacaagtgcctcaaaaattcttcgctgttaaaaacctgctgcttctcccaggctcctacacttatgagtggaactttaggaaggatgtgaacatggttctacagagttccctcggtaacgacctgcgggtagatggcgccagcatcagtttcacgagcatcaacctctatgctacttttttccccatggctcacaacaccgcttccacccttgaagccatgctgcggaatgacaccaatgatcagtcattcaacgactacctatctgcagctaacatgctctaccccattcctgccaatgcaaccaatattcccatttccattccttctcgcaactgggcggctttcagaggctggtcatttaccagactgaaaaccaaagaaactccctctttggggtctggatttgacccctactttgtctattctggttctattccctacctggatggtaccttctacctgaaccacacttttaagaaggtttccatcatgtttgactcttcagtgagctggcctggaaatgacaggttactatctcctaacgaatttgaaataaagcgcactgtggatggcgaaggctacaacgtagcccaatgcaacatgaccaaagactggttcttggtacagatgctcgccaactacaacatcggctatcagggcttctacattccagaaggatacaaagatcgcatgtattcatttttcagaaacttccagcccatgagcaggcaggtggttgatgaggtcaattacaaagacttcaaggccgtcgccataccctaccaacacaacaactctggctttgtgggttacatggctccgaccatgcgccaaggtcaaccctatcccgctaactatccctatccactcattggaacaactgccgtaaatagtgttacgcagaaaaagttcttgtgtgacagaaccatgtggcgcataccgttctcgagcaacttcatgtctatgggggcccttacagacttgggacagaatatgctctatgccaactcagctcatgctctggacatgacctttgaggtggatcccatggatgagcccaccctgctttatcttctcttcgaagttttcgacgtggtcagagtgcatcagccacaccgcggcatcatcgaggcagtctacctgcgtacaccgttctcggccggtaacgctaccacgtaagaagcttcttgcttcttgcaaatagcagctgcaaccatggcctgcggatcccaaaacggctccagcgagcaagagctcagagccattgtccaagacctgggttgcggaccctattttttgggaacctacgataagcgcttcccggggttcatggcccccgataagctcgcctgtgccattgtaaatacggccggacgtgagacggggggagagcactggttggctttcggttggaacccacgttctaacacctgctacctttttgatccttttggattctcggatgatcgtctcaaacagatttaccagtttgaatatgagggtctcctgcgccgcagcgctcttgctaccaaggaccgctgtattacgctggaaaaatctacccagaccgtgcagggtccccgttctgccgcctgcggacttttctgctgcatgttccttcacgcctttgtgcactggcctgaccgtcccatggacggaaaccccaccatgaaattgctaactggagtgccaaacaacatgcttcattctcctaaagtccagcccaccctgtgtgacaatcaaaaagcactctaccattttcttaatacccattcgccttattttcgctcccatcgtacacacatcgaaagggccactgcgttcgaccgtatggatgttcaataatgactcatgtaaacaacgtgttcaataaacatcactttatttttttacatgtatcaaggctctgcattacttatttatttacaagtcgaatgggttctgacgagaatcagaatgacccgcaggcagtgatacgttgcggaactgatacttgggttgccacttgaattcgggaatcaccaacttgggaaccggtatatcgggcaggatgtcactccacagctttctggtcagctgcaaagctccaagcaggtcaggagccgaaatcttgaaatcacaattaggaccagtgctttgagcgcgagagttgcggtacaccggattgcagcactgaaacaccatcagcgacggatgtctcacgcttgccagcacggtgggatctgcaatcatgcccacatccagatcttcagcattggcaatgctgaacggggtcatcttgcaggtctgcctacccatggcgggcacccaattaggcttgtggttgcaatcgcagtgcagggggatcagtatcatcttggcctgatcctgtctgattcctggatacacggctctcatgaaagcatcatattgcttgaaagcctgctgggctttactaccctcggtataaaacatcccgcaggacctgctcgaaaactggttagctgcacagccggcatcattcacacagcagcgggcgtcattgttagctatttgcaccacacttctgccccagcggttttgggtgattttggttcgctcgggattctcctttaaggctcgttgtccgttctcgctggccacatccatctcgataatctgctccttctgaatcataatattgccatgcaggcacttcagcttgccctcataatcattgcagccatgaggccacaacgcacagcctgtacattcccaattatggtgggcgatctgagaaaaagaatgtatcattccctgcagaaatcttcccatcatcgtgctcagtgtcttgtgactagtgaaagttaactggatgcctcggtgctcctcgtttacgtactggtgacagatgcgcttgtattgttcgtgttgctcaggcattagtttaaaagaggttctaagttcgttatccagcctgtacttctccatcagcagacacatcacttccatgcctttctcccaagcagacaccaggggcaagctaatcggattcttaacagtgcaggcagcagctcctttagccagagggtcatctttagcgatcttctcaatgcttcttttgccatccttctcaacgatgcgcacgggcgggtagctgaaacccactgctacaagttgcgcctcttctctttcttcttcgctgtcttgactgatgtcttgcatggggatatgtttggtcttccttggcttctttttggggggtatcggaggaggaggactgtcgctccgttccggagacagggaggattgtgacgtttcgctcaccattaccaactgactgtcggtagaagaacctgaccccacacggcgacaggtgtttctcttcgggggcagaggtggaggcgattgcgaagggctgcggtccgacctggaaggcggatgactggcagaaccccttccgcgttcgggggtgtgctccctgtggcggtcgcttaactgatttccttcgcggctggccattgtgttctcctaggcagagaaacaacagacatggaaactcagccattgctgtcaacatcgccacgagtgccatcacatctcgtcctcagcgacgaggaaaaggagcagagcttaagcattccaccgcccagtcctgccaccacctctaccctagaagataaggaggtcgacgcatctcatgacatgcagaataaaaaagcgaaagagtctgagacagacatcgagcaagacccgggctatgtgacaccggtggaacacgaggaagagttgaaacgctttctagagagagaggatgaaaactgcccaaaacaacgagcagataactatcaccaagatgctggaaatagggatcagaacaccgactacctcatagggcttgacggggaagacgcgctccttaaacatctagcaagacagtcgctcatagtcaaggatgcattattggacagaactgaagtgcccatcagtgtggaagagctcagccgcgcctacgagcttaacctcttttcacctcgtactccccccaaacgtcagccaaacggcacctgcgagccaaatcctcgcttaaacttttatccagcttttgctgtgccagaagtactggctacctatcacatcttttttaaaaatcaaaaaattccagtctcctgccgcgctaatcgcacccgcgccgatgccctactcaatctgggacctggttcacgcttacctgatatagcttccttggaagaggttccaaagatcttcgagggtctgggcaataatgagactcgggccgcaaatgctctgcaaaagggagaaaatggcatggatgagcatcacagcgttctggtggaattggaaggcgataatgccagactcgcagtactcaagcgaagcatcgaggtcacacacttcgcatatcccgctgtcaacctgccccctaaagtcatgacggcggtcatggaccagttactcattaagcgcgcaagtcccctttcagaagacatgcatgacccagatgcctgtgatgagggtaaaccagtggtcagtgatgagcagctaacccgatggctgggcaccgactctcccagggatttggaagagcgtcgcaagcttatgatggccgtggtgctggttaccgtagaactagagtgtctccgacgtttctttaccgattcagaaaccttgcgcaaactcgaagagaatctgcactacacttttagacacggctttgtgcggcaggcatgcaagatatctaacgtggaactcaccaacctggtttcctacatgggtattctgcatgagaatcgcctaggacaaagcgtgctgcacagcaccctgaagggggaagcccgccgtgattacatccgcgattgtgtctatctgtacctgtgccacacgtggcaaaccggcatgggtgtatggcagcaatgtttagaagaacagaacttgaaagagcttgacaagctcttacagaaatctcttaaggttctgtggacagggttcgacgagcgcaccgtcgcttccgacctggcagacctcatcttcccagagcgtctcagggttactttgcgaaacggattgcctgactttatgagccagagcatgcttaacaattttcgctctttcatcctggaacgctccggtatcctgcccgccacctgctgcgcactgccctccgactttgtgcctctcacctaccgcgagtgccccccgccgctatggagtcactgctacctgttccgtctggccaactatctctcctaccactcggatgtgatcgaggatgtgagcggagacggcttgctggagtgtcactgccgctgcaatctgtgcacgccccaccggtccctagcttgcaacccccagttgatgagcgaaacccagataataggcacctttgaattgcaaggccccagcagccaaggcgatgggtcttctcctgggcaaagtttaaaactgaccccgggactgtggacctccgcctacttgcgcaagtttgctccggaagattaccacccctatgaaatcaagttctatgaggaccaatcacagcctccaaaggccgaactttcggcctgcgtcatcacccagggggcaattctggcccaattgcaagccatccaaaaatcccgccaagaatttctactgaaaaagggtaagggggtctaccttgacccccagaccggcgaggaactcaacacaaggttccctcaggatgtcccaacgacgagaaaacaagaagttgaaggtgcagccgccgcccccagaagatatggaggaagattgggacagtcaggcagaggaggcggaggaggacagtctggaggacagtctggaggaagacagtttggaggaggaaaacgaggaggcagaggaggtggaagaagtaaccgccgacaaacagttatcctcggctgcggagacaagcaacagcgctaccatctccgctccgagtcgaggaacccggcggcgtcccagcagtagatgggacgagaccggacgcttcccgaacccaaccagcgcttccaagaccggtaagaaggatcggcagggatacaagtcctggcgggggcataagaatgccatcatctcctgcttgcatgagtgcgggggcaacatatccttcacgcggcgctacttgctattccaccatggggtgaactttccgcgcaatgttttgcattactaccgtcacctccacagcccctactatagccagcaaatcccggcagtctcgacagataaagacagcggcggcgacctccaacagaaaaccagcagcggcagttagaaaatacacaacaagtgcagcaacaggaggattaaagattacagccaacgagccagcgcaaacccgagagttaagaaatcggatctttccaaccctgtatgccatcttccagcagagtcggggtcaagagcaggaactgaaaataaaaaaccgatctctgcgttcgctcaccagaagttgtttgtatcacaagagcgaagatcaacttcagcgcactctcgaggacgccgaggctctcttcaacaagtactgcgcgctgactcttaaagagtaggcagcgaccgcgcttattcaaaaaaggcgggaattacatcatcctcgacatgagtaaagaaattcccacgccttacatgtggagttatcaaccccaaatgggattggcggcaggcgcctcccaggactactccacccgcatgaattggctcagcgccgggccttctatgatttctcgagttaatgatatacgcgcctaccgaaaccaaatacttttggaacagtcagctcttaccaccacgccccgccaacaccttaatcccagaaattggcccgccgccctagtgtaccaggaaagtcccgctcccaccactgtattacttcctcgagacgcccaggccgaagtccaaatgactaatgcaggtgcgcagttagctggcggctccaccctatgtcgtcacaggcctcggcataatataaaacgcctgatgatcagaggccgaggtatccagctcaacgacgagtcggtgagctctccgcttggtctacgaccagacggaatctttcagattgccggctgcgggagatcttccttcacccctcgtcaggctgttctgactttggaaagttcgtcttcgcaaccccgctcgggcggaatcgggaccgttcaatttgtggaggagtttactccctctgtctacttcaaccccttctccggatctcctgggcattacccggacgagttcataccgaacttcgacgcgattagcgagtcagtggacggctacgattgatgtctggtgacgcggctgagctatctcggctgcgacatctagaccactgccgccgctttcgctgctttgcccgggaactcattgagttcatctacttcgaactccccaaggatcaccctcaaggtccggcccacggagtgcggatttctatcgaaggcaaaatagactctcgcctgcaacgaattttctcccagcggcccgtgctgatcgagcgagaccagggaaacaccacggtttccatctactgcatttgtaatcaccccggattgcatgaaagcctttgctgtcttatgtgtactgagtttaataaaaactgaattaagactctcctacggactgccgcttcttcaacccggattttacaaccagaagaacgaaacttttcctgtcgtccaggactctgttaacttcacctttcctactcacaaactagaagctcaacgactacaccgcttttccagaagcattttccctactaatactactttcaaaaccggaggtgagctccaaggtcttcctacagaaaacccttgggtggaagcgggccttgtagtgctaggaattcttgcgggtgggcttgtgattattctttgctacctatacacaccttgcttcactttcttagtggtgttgtggtattggtttaaaaaatggggcccatactagtcttgcttgttttactttcgcttttggaaccgggttctgccaattacgatccatgtctagacttcgacccagaaaactgcacacttacttttgcacccgacacaagccgcatctgtggagttcatcgcctctcttacgaacttggcccccaacgacaaaaatttacctgcatggtgggaatcaaccccatagttatcacccagcaaagtggagatactaagggttgcattcactgctcctgcgattccatcgagtgcacctacaccctgctgaagaccctatgcggcctaagagacctgctaccaatgaattaaaaaatgattaataaaaaatcacttacttgaaatcagcaataaggtctctgttgaaattttctcccagcagcacctcacttccctcttcccaactctggtattctaaaccccgttcagcggcatactttctccatactttaaaggggatgtcaaattttagctcctctcctgtacccacaatcttcatgtctttcttcccagatgaccaagagagtccggctcagtgactccttcaaccctgtctacccctatgaagatgaaagcacctcccaacacccctttataaacccagggtttatttccccaaatggcttcacacaaagcccaaacggagttcttactttaaaatgtttaaccccactaacaaccacaggcggatctctacagctaaaagtgggagggggacttacagtggatgacaccaacggttttttgaaagaaaacataagtgccaccacaccactcgttaagactggtcactctataggtttaccactaggagccggattgggaacgaatgaaaataaactttgtatcaaattaggacaaggacttacattcaattcaaacaacatttgcattgatgacaatattaacaccttatggacaggagtcaaccccaccgaagccaactgtcaaatcatgaactccagtgaatctaatgattgcaaattaattctaacactagttaaaactggagcactagtcactgcatttgtttatgttataggagtatctaacaattttaatatgctaactacacacagaaatataaattttactgcagagctgtttttcgattctactggtaatttactaactagactctcatccctcaaaactccacttaatcataaatcaggacaaaacatggctactggtgccattactaatgctaaaggtttcatgcccagcacgactgcctatcctttcaatgataattctagagaaaaagaaaactacatttacggaacttgttactacacagctagtgatcgcactgcttttcccattgacatatctgtcatgcttaaccgaagagcaataaatgacgagacatcatattgtattcgtataacttggtcctggaacacaggagatgccccagaggtgcaaacctctgctacaaccctagtcacctccccatttaccttttactacatcagagaagacgactgacaaataaagtttaacttgtttatttgaaaatcaattcacaaaatccgagtagttattttgcctcccccttcccatttaacagaatacaccaatctctccccacgcacagctttaaacatttggataccattagatatagacatggttttagattccacattccaaacagtttcagagcgagccaatctggggtcagtgatagataaaaatccatcgggatagtcttttaaagcgctttcacagtccaactgctgcggatgcgactccggagtctggatcacggtcatctggaagaagaacgatgggaatcataatccgaaaacggtatcggacgattgtgtctcatcaaacccacaagcagccgctgtctgcgtcgctccgtgcgactgctgtttatgggatcagggtccacagtgtcctgaagcatgattttaatagcccttaacatcaactttctggtgcgatgcgcgcagcaacgcattctgatttcactcaaatctttgcagtaggtacaacacattattacaatattgtttaataaaccataattaaaagcgctccagccaaaactcatatctgatataatcgcccctgcatgaccatcataccaaagtttaatataaattaaatgacgttccctcaaaaacacactacccacatacatgatctcttttggcatgtgcatattaacaatctgtctgtaccatggacaacgttggttaatcatgcaacccaatataaccttccggaaccacactgccaacaccgctcccccagccatgcattgaagtgaaccctgctgattacaatgacaatgaagaacccaattctctcgaccgtgaatcacttgagaatgaaaaatatctatagtggcacaacatagacataaatgcatgcatcttctcataatttttaactcctcaggatttagaaacatatcccagggaataggaagctcttgcagaacagtaaagctggcagaacaaggaagaccacgaacacaacttacactatgcatagtcatagtatcacaatctggcaacagcgggtggtcttcagtcatagaagctcgggtttcattttcctcacaacgtggtaactgggctctggtgtaagggtgatgtctggcgcatgatgtcgagcgtgcgcgcaaccttgtcataatggagttgcttcctgacattctcgtattttgtatagcaaaacgcggccctggcagaacacactcttcttcgccttctatcctgccgcttagcgtgttccgtgtgatagttcaagtacaaccacactcttaagttggtcaaaagaatgctggcttcagttgtaatcaaaactccatcgcatctaatcgttctgaggaaatcatccaagcaatgcaactggattgtgtttcaagcaggagaggagagggaagagacggaagaaccatgttaatttttattccaaacgatctcgcagtacttcaaattgtagatcgcgcagatggcatctctcgcccccactgtgttggtgaaaaagcacagctagatcaaaagaaatgcgattttcaaggtgctcaacggtggcttccagcaaagcctccacgcgcacatccaagaacaaaagaataccaaaagaaggagcattttctaactcctcaatcatcatattacattcctgcaccattcccagataattttcagctttccagccttgaattattcgtgtcagttcttgtggtaaatccaatccacacattacaaacaggtcccggagggcgccctccaccaccattcttaaacacaccctcataatgacaaaatatcttgctcctgtgtcacctgtagcgaattgagaatggcaacatcaattgacatgcccttggctctaagttcttctttaagttctagttgtaaaaactctctcatattatcaccaaactgcttagccagaagccccccgggaacaagagcaggggacgctacagtgcagtacaagcgcagacctccccaattggctccagcaaaaacaagattggaataagcatattgggaaccgccagtaatatcatcgaagttgctggaaatataatcaggcagagtttcttgtaaaaattgaataaaagaaaaatttgccaaaaaaacattcaaaacctctgggatgcaaatgcaataggttaccgcgctgcgctccaacattgttagttttgaattagtctgcaaaaataaaaaaaaaaacaagcgtcatatcatagtagcctgacgaacagatggataaatcagtctttccatcacaagacaagccacagggtctccagctcgaccctcgtaaaacctgtcatcatgattaaacaacagcaccgaaagttcctcgcggtgaccagcatgaataattcttgatgaagcatacaatccagacatgttagcatcagttaacgagaaaaaacagccaacatagcctttgggtataattatgcttaatcgtaagtatagcaaagccacccctcgcggatacaaagtaaaaggcacaggagaataaaaaatataattatttctctgctgctgttcaggcaacgtcgcccccggtccctctaaatacacatacaaagcctcatcagccatggcttaccagacaaagtacagcgggcacacaaagcacaagctctaaagtgactctccaacctctccacaatatatatatacacaagccctaaactgacgtaatgggagtaaagtgtaaaaaatcccgccaaacccaacacacaccccgaaactgcgtcaccagggaaaagtacagtttcacttccgcaatcccaacaggcgtaacttcctctttctcacggtacgtgatatcccactaacttgcaacgtcattttcccacggtcgcaccgccccttttagccgttaaccccacagccaatcaccacacgatccacactttttaaaatcacctcatttacatattggcaccattccatctataaggtatattatatagataga

SEQ ID NO: 2 - DNA, 5287 bp, Artificial Sequence
  ColoAd1 chimeric E2B
ctatggcatctcgatccagcagacctcctcgtttcgcgggtttggacggctcctggaatagggtatgagacgatgggcgtccagcgctgccagggttcggtccttccagggtctcagtgttcgagtcagggttgtttccgtcacagtgaaggggtgtgcgcctgcttgggcgcttgccagggtgcgcttcagactcatcctgctggtcgaaaacttctgtcgcttggcgccctgtatgtcggccaagtagcagtttaccatgagttcgtagttgagcgcctcggctgcgtggcctttggcgcggagcttacctttggaagttttcttgcataccgggcagtataggcatttcagcgcatacaacttgggcgcaaggaaaacggattctggggagtatgcatctgcgccgcaggaggcgcaaacagtttcacattccaccagccaggttaaatccggttcattggggtcaaaaacaagttttccgccatattttttgatgcgtttcttacctttggtctccatgagttcgtgtcctcgttgagtgacaaacaggctgtccgtgtccccgtagactgattttacaggcctcttctccagtggagtgcctcggtcttcttcgtacaggaactctgaccactctgatacaaaggcgcgcgtccaggccagcacaaaggaggctatgtgggaggggtagcgatcgttgtcaaccagggggtccaccttttccaaagtatgcaaacacatgtcaccctcttcaacatccaggaatgtgattggcttgtaggtgtatttcacgtgacctggggtccccgctgggggggtataaaagggggcggttctttgctcttcctcactgtcttccggatcgctgtccaggaacgtcagctgttggggtaggtattccctctcgaaggcgggcatgacctctgcactcaggttgtcagtttctaagaacgaggaggatttgatattgacagtgccggttgagatgcctttcatgaggttttcgtccatctggtcagaaaacacaatttttttattgtcaagtttggtggcaaatgatccatacagggcgttggataaaagtttggcaatggatcgcatggtttggttcttttccttgtccgcgcgctctttggcggcgatgttgagttggacatactcgcgtgccaggcacttccattcggggaagatagttgttaattcatctggcacgattctcacttgccaccctcgattatgcaaggtaattaaatccacactggtggccacctcgcctcgaaggggttcattggtccaacagagcctacctcctttcctagaacagaaagggggaagtgggtctagcataagttcatcgggagggtctgcatccatggtaaagattcccggaagtaaatccttatcaaaatagctgatgggagtggggtcatctaaggccatttgccattctcgagctgccagtgcgcgctcatatgggttaaggggactgccccatggcatgggatgggtgagtgcagaggcatacatgccacagatgtcatagacgtagatgggatcctcaaagatgcctatgtaggttggatagcatcgcccccctctgatacttgctcgcacatagtcatatagttcatgtgatggcgctagcagccccggacccaagttggtgcgattgggtttttctgttctgtagacgatctggcgaaagatggcgtgagaattggaagagatggtgggtctttgaaaaatgttgaaatgggcatgaggtagacctacagagtctctgacaaagtgggcataagattcttgaagcttggttaccagttcggcggtgacaagtacgtctagggcgcagtagtcaagtgtttcttgaatgatgtcataacctggttggtttttcttttcccacagttcgcggttgagaaggtattcttcgcgatccttccagtactcttctagcggaaacccgtctttgtctgcacggtaagatcctagcatgtagaactgattaactgccttgtaagggcagcagcccttctctacgggtagagagtatgcttgagcagcttttcgtagcgaagcgtgagtaagggcaaaggtgtctctgaccatgactttgaggaattggtatttgaagtcgatgtcgtcacaggctccctgttcccagagttggaagtctacccgtttcttgtaggcggggttgggcaaagcgaaagtaacatcattgaagagaatcttgccggccctgggcatgaaattgcgagtgatgcgaaaaggctgtggtacttccgctcggttattgataacctgggcagctaggacgatctcgtcgaaaccgttgatgttgtgtcctacgatgtataattctatgaaacgcggcgtgcctctgacgtgaggtagcttactgagctcatcaaaggttaggtctgtggggtcagataaggcgtagtgttcgagagcccattcgtgcaggtgaggattcgctttaaggaaggaggaccagaggtccactgccagtgctgtttgtaactggtcccggtactgacgaaaatgccgtccgactgccattttttctggggtgacgcaatagaaggtttgggggtcctgccgccagcgatcccacttgagttttatggcgaggtcataggcgatgttgacgagccgctggtctccagagagtttcatgaccagcatgaaggggattagctgcttgccaaaggaccccatccaggtgtaggtttccacatcgtaggtgagaaagagcctttctgtgcgaggatgagagccaatcgggaagaactggatctcctgccaccagttggaggaatggctgttgatgtgatggaagtagaactccctgcgacgcgccgagcattcatgcttgtgcttgtacagacggccgcagtagtcgcagcgttgcacgggttgtatctcgtgaatgagttgtacctggcttcccttgacgagaaatttcagtgggaagccgaggcctggcgattgtatctcgtgctttactatgttgtctgcatcggcctgttcatcttctgtctcgatggtggtcatgctgacgagccctcgcgggaggcaagtccagacctcggcgcggcaggggcggagctcgaggacgagagcgcgcaggctggagctgtccagggtcctgagacgctgcggactcaggttagtaggcagtgtcaggagattaacttgcatgatcttttggagggcgtgcgggaggttcagatagtacttgatctcaacgggtccgttggtggagatgtcgatggcttgcagggttccgtgtcccttgggcgctaccaccgtgcccttgtttttcattttggacggcggtggctctgttgcttcttgcatgtttagaagcggtgtcgagggcgcgcaccgggcggcaggggcggctcgggacccggcggcatggctggcagtggtacgtcggcgccgcgcgcgggtaggttctggtactgcgccctgagaagactcgcatgcgcgacgacgcggcggttgacatcctggatctgacgcctctgggtgaaagctaccggccccgtgagcttgaacctgaaagagagttcaacagaatcaatctcggtatcgttgacggcggcttgcctaaggatttcttgcacgtcaccagagttgtcctggtaggcgatctccgccatgaactgctcgatctcttcctcttgaagatctccgcggcccgctctctcgacggtggccgcgaggtcgttggagatgcgcccaatgagttgagagaatgcattcatgcccgcctcgttccagacgcggctgtagaccacggcccccacgggatctctcgcgcgcatgaccacctgggcgaggttgagctccacgtggcgggtgaagaccgcatagttgcataggcgctggaaaaggtagttgagtgtggtggcgatgtgctcggtgacgaagaaatacatgatccatcgtctcagcggcatctcgctgacatcgcccagagcttccaagcgctccatggcctcgtagaagtccacggcaaaattaaaaaactgggagtttcgcgcggacacggtcaactcctcttccagaagacggataagttcggcgatggtggtgcgcacctcgcgctcgaaagcccctgggatttcttcctcaatctcttcttcttccactaacatctcttcctcttcaggtggggctgcaggaggagggggaacgcggcgacgccggcggcgcacgggcagacggtcgatgaatctttcaatgacctctccgcggcggcggcgcatggtttcagtgacggcgcggccgttctcgcgcggtcgcagagtaaaaacaccgccgcgcatctccttaaagtggtgactgggaggttctccgtttgggagggagagggcgctgattatacattttattaattggcccgtagggactgcacgcagagatctgatcgtgtcaagatccacgggatctgaaaacctttcgacgaaagcgtctaaccagtcacagtcacaaggtaggctgagtacggcttcttgtgggcgggggtggttatgtgttcggtctgggtcttctgtttcttcttcatctcgggaaggtgagacgatgctgctggtgatgaaattaaagtaggcagttctaagacggcggatggtggcgaggagcaccaggtctttgggtccggcttgctggatacgcaggcgattggccattccccaagcattatcctgacatctagcaagatctttgtagtagtcttgcatgagccgttctacgggcacttcttcctcacccgttctgccatgcatacgtgtgagtccaaatccgcgcattggttgtaccagtgccaagtcagctacgactctttcggcgaggatggcttgctgtacttgggtaagggtggcttgaaagtcatcaaaatccacaaagcggtggtaagctcctgtattaatggtgtaagcacagttggccatgactgaccagttaactgtctggtgaccagggcgcacgagctcggtgtatttaaggcgcgaataggcgcgggtgtcaaagatgtaatcgttgcaggtgcgcaccagatactggtaccctataagaaaatgcggcggtggttggcggtagagaggccatcgttctgtagctggagcgccaggggcgaggtcttccaacataaggcggtgatagccgtagatgtacctggacatccaggtgattcctgcggcggtagtagaagcccgaggaaactcgcgtacgcggttccaaatgttgcgtagcggcatgaagtagttca